(19) United States
(12) Patent Application Publication
(10) Pub. No.: US 2009/0246142 A1
(43) Pub. Date: Oct. 1, 2009

(54) Triggered Self-Assembly of Nanoparticles In Vivo

(75) Inventors: Sangeeta N. Bhatia, Lexington, MA (US); Todd J. Harris, Winthrop, MA (US); Geoffrey von Maltzahn, Boston, MA (US)

Correspondence Address: CHOATE, HALL & STEWART LLP, TWO INTERNATIONAL PLACE, BOSTON, MA, 02110, US

(73) Assignee: MASSACHUSETTS INSTITUTE OF TECHNOLOGY, Cambridge, MA (US)

(21) Appl. No.: 12/282,462

(22) PCT Filed: Mar. 9, 2007
(86) PCT No.: PCT/US07/06141

§ 371 Date: Mar. 16, 2009

Related U.S. Application Data
(60) Provisional application No. 60/780,959, filed on Mar. 10, 2006.

(51) Int. Cl. A61K 49/00; A61K 9/127; A61K 9/14; A61K 39/395; A61K 31/7088; A61K 38/02

(52) U.S. Cl. 424/9.4; 424/450; 424/489; 424/178.1; 514/44 R; 514/2; 424/9.1; 424/9.5

(57) ABSTRACT

The present invention provides triggered self-assembly nanosystems. Such nanosystems comprise a population of triggered self-assembly conjugates, each conjugate comprising one or more monomeric units and one or more complementary binding moieties. In some embodiments, inventive nanosystems and conjugates can be used to treat and/or diagnose a disease, disorder, and/or condition.

### RELATED APPLICATION

The present application is related to and claims priority under 35 U.S.C. § 119(e) to U.S. Ser. No. 60/780,959, filed Mar. 10, 2006 (the '959 application). The entire contents of the '959 application are incorporated herein by reference.

### GOVERNMENT SUPPORT

The United States Government has provided grant support utilized in the development of the present invention. In particular, National Cancer Institute/NASA contract number N01-CO37117 has supported development of this invention. The United States Government may have certain rights in the invention.

### BACKGROUND OF THE INVENTION

The current practice of therapeutic and diagnostic targeting involves the attachment of a targeting moiety (e.g., antibody, peptide, etc.) to a cargo of interest. The efficacy of such a conjugate for therapy or diagnosis is determined both by the specificity of the targeting moiety (i.e., the concentration in target tissue versus background) and by the quantity of conjugate delivered to the target. Because increasing specificity typically decreases yield, these two goals are often mutually exclusive, resulting in either significant collateral toxicity and background signal or in target accumulation below effective therapeutic or diagnostic limits.

Current methods for targeted therapeutics and diagnostics include ligand-targeting, passive targeting, externally directed activation of therapeutic, and/or biochemical directed activation for targeting. In ligand-targeting methods, toxins, drugs, activators, or nanomaterial cargoes are typically conjugated to peptide ligands or antibodies, which direct the cargo to the desired site (Allen, 2002, Nature Rev. Drug Discov., 2:750). In this case, uptake by reticulo-endothelial system (RES) or non-specific association of ligands or antibodies with other proteins of serum, extracellular matrix, or membrane often limits the efficacy of this method (Moghimi et al., 2001, Pharmacol. Rev., 53:283).

Passive targeting techniques generally rely on increased extravasation through leaky vessels at a target site. Long circulating polymers, liposomes, or nanoparticles are directed to a target through passive accumulation, an effect known as enhanced permeability and retention (EPR) (Mastsumura et al., 1986, Cancer Res., 6:6387). This strategy, primarily used in tumor targeting, is limited by the heterogenous structure of tumor tissue including areas of necrosis, high interstitial pressure, and little to no perfusion (Hobbs et al., 1998, Proc. Natl. Acad. Sci., USA, 95:4607).

Alternative approaches for targeted delivery rely on external triggers to activate or deliver therapeutic agent to a diseased site. For example, focused ultrasound can be used to burst “microbubbles” to release encapsulated drug or toxin at a desired site (Pruitt et al., 2002, Drug Deliv., 9:253). This technique is limited by the short half-life of microbubbles in the blood. External light irradiation of porphyrin, drug, or nanomaterial can be used to activate a therapeutic or generate a free radical form of oxygen for photodynamic therapy (PDT) at a site (see e.g., US Patent Publication 2003/0208249). The low wavelength light necessary to activate the free radical chemistry has poor transmission through tissue, thus insertion of probes surgically is used to activate PDT chemistries in deep tissues. Near-infrared illumination of plasmon resonant nanoshells can be used to ablate tumors through heating (West et al., 2003, Ann. Rev. Biomed. Eng., 5:285). Near-infrared light is more transparent to the body than other wavelengths, but is still attenuated on the order of a few centimeters, limiting the efficacy of this treatment in deep tissues.

Biochemical triggers have been demonstrated for target specific triggering of a therapeutic. pH-sensitive, lipid-anchored copolymers and protease-cleavable PEG chains have been incorporated into liposomes to generate vesicles that are stable under normal conditions, but become unstable when activated by their biochemical trigger (Drummond, et al., 1999, Pharmacol Rev., 51:691). Activation of liposomes leads to fusion and incorporation into cellular membranes. This technique has been employed to generate liposomes capable of routing their contents out of the endosome and into the cytosol (Meyer, et al., 1998, FEBS Lett., 421:61), or directly into the cell membrane into the cytosol (Kirpotin, et al., 1996, FEBS Lett., 388:115; and Zalipsky, et al., 1997, Bioconjugate Chem., 10:703). This technique is limited in its versatility as it is only relevant to liposomal fusion.

Protease activation has been used to increase the internalization of a cargo through unmasking of a fused TAT-like peptide domain (Jiang, et al., 2004, Proc. Natl. Acad. Sci., USA, 101:17867). Masking is accomplished through a negatively charged cleavable peptide that neutralizes the positive charge of a TAT-like domain. Upon arrival to a tumor, the negatively charged domain is cleaved by a protease and the remaining TAT-like domain associates with the cell membrane to facilitate its internalization to cells at the tumor site. This technique has been demonstrated with a single peptide and with a small molecule cargo. More recently, this technique has been demonstrated with nanoparticles and utilizes charge neutralization (i.e. anions on the end of a cationic sequence) as opposed to some form of steric shielding (Zhang et al., 2006, Nano Lett., 6:1988).

Protease activation has been used to release near infrared (NIR) probes from their quenched state on the backbone of poly-lysine or nanoparticle substrate (Mahmood et al., 2003, Mol. Cancer. Ther., 2:489). Upon activation, NIR fluorescence increases several fold, enabling detection of diseased areas in which proteases are upregulated. Protease-mediated activation of a photodynamic agent has been used to extend this technology to the therapeutic regime (Choi et al., 2004, Bioconj. Chem., 15:242); however, this technology utilizes disassembly in order to enhance fluorescence; thus, this system cannot be applied to materials that have gain-of-function or enhanced properties due to assembly as opposed to disassembly.

Self-assembly of nanomaterials has been used to accomplish very sensitive detection, primarily in vitro. Attomolar detection of DNA has been demonstrated in pure samples using gold nanoparticles modified with complementary DNA strands (Mirkin, et al., 1996, Nature, 382:607). Assembly of gold nanoparticles leads to an absorption and light scattering shift due to plasmon resonance shifts from closely assembled particles. Sensitive detection has been demonstrated with self-assembling iron oxide nanoparticles (Perez, et al., 2002, Nat. Biotechnol., 20:816). The close proximity of iron-oxide nanoparticles in an assembled construct changes T2 relaxivity of the surrounding media, giving a detectable T2 weighted signal reduction in an MRI. Assembly of iron-oxide nanoparticles around a virus for in vitro detection as well as peroxidase activated aggregation of nanoparticles in solution has been demonstrated (Perez, et al., 2003, J. Am. Chem. Soc., 25:10192; and Bogdanov, et al., 2002, Mol. Imaging, 1: 16).

Thus, there is a need for therapeutic and diagnostic methods that are highly specific, highly potent, capable of functioning in deep tissues, and able to avoid clearance by the kidney. There is a strong need for methods that allow for controlled temporal and spatial delivery of therapeutic and/or diagnostic agents to a particular organ, tissue, cell, intracellular compartment, etc.

### SUMMARY OF THE INVENTION

The present invention provides methods of triggering self-assembly of individual components (e.g., nanoparticles, microparticles, dendrimers, nanoemulsions, liposomes, polymers, micelles, proteins, peptides, and/or other monomeric units) at or near an in vivo or in vitro target for diagnostic and/or therapeutic purposes. In some embodiments, the individual components are complementary objects. Such methods comprise conjugating monomeric units with complementary binding moieties which mediate self-assembly to generate triggered self-assembly conjugates (TSACs). Such methods optionally comprise modifying a TSAC with one or more blocking agents which prevent self-assembly in an initial state, but upon removal, actuate TSAC self-assembly.

The present invention provides conjugates comprising a biologically compatible monomeric unit and at least one complementary binding moiety conjugated to the monomeric unit. Any substance to which complementary binding moieties can be attached may act as a monomeric unit according to the present invention. In some embodiments, a monomeric unit is selected from the group consisting of a nanoparticle, microparticle, dendrimer, nanoemulsion, liposome, polymer, micelle, protein, peptide, etc. In certain specific embodiments, the monomeric unit is a nanoparticle.

A complementary binding moiety can be any binding moiety capable of interacting with a cognate at a desired location or under desired conditions. For example, complementary binding moieties can be ligands and anti-ligands (e.g. streptavidin and biotin), ligands and receptors (e.g. small molecule ligands and their receptors), antibodies and antigens, phage display-derived peptides, complementary nucleic acids (e.g. DNA hybrids, RNA hybrids, DNA/RNA hybrids, etc.), and aptamers. Other exemplary complementary binding moieties include, but are not limited to, moieties exhibiting complementary charges, hydrophobicity, hydrogen bonding, covalent bonding, Van der Waals forces, reactive chemistries, electrostatic interactions, magnetic interactions, etc. In some embodiments, complementary binding moieties include streptavidin and biotin.

In some embodiments, inventive conjugates may optionally comprise at least one removably associated blocking agent, wherein the blocking agent shields the complementary binding moiety until the blocking agent is removed. Any polymeric entity can serve as a blocking agent in accordance with the present invention. In some embodiments, a blocking agent can include polaxamines; poloxamers; polyethylene glycol (PEG); peptides; synthetic polymers of sufficient length and density to both mask self-assembly and provide protection against non-specific adsorption, opsonization, and RES uptake; and/or combinations thereof.

In some embodiments, a blocking agent is conjugated to a complementary binding moiety or to a monomeric unit by a cleavable linker. Cleavable linkers of the invention may be selected to be cleaved via any form of cleavable chemistry. Exemplary cleavable linkers include, but are not limited to, protease cleavable peptide linkers, nuclease sensitive nucleic acid linkers, lipase sensitive lipid linkers, glycosidase sensitive carbohydrate linkers, pH sensitive linkers, hypoxia sensitive linkers, photo-cleavable linkers, heat-labile linkers, enzyme cleavable linkers, ultrasound-sensitive linkers, x-ray cleavable linkers, etc.

In some embodiments, self-assembly of TSACs provides one or more properties which are displayed only upon self-assembly of TSACs, but are not displayed when TSACs are separate and have not self-assembled. In some embodiments, self-assembly of monomeric units provides one or more emergent properties. Emergent properties may be electrical, magnetic, optical, mechanical, and/or biological. In some embodiments, emergent properties can be assayed and/or measured.

In some embodiments, TSAC self-assembly provides an emergent property by bringing together two or more “cargo entities” which are conjugated to the TSAC. In some embodiments, a cargo entity is a diagnostic and/or therapeutic agent to be delivered. In some embodiments, a cargo entity is a substance that does not require TSAC self-assembly to be active and/or effective. In some embodiments, such a cargo entity may be conjugated to a TSAC and made available to a target site only upon self-assembly of the TSACs to which the cargo entity is conjugated. In some embodiments, a cargo entity is a substance that, by itself, has little to no desired effect. However, upon TSAC aggregation, cargo entities can interact to achieve a desired result (e.g. emergent property, as described herein).

The invention provides a triggered self-assembly nanosystem (TSAN), comprising one or more populations of individual TSACs. In some embodiments, an inventive TSAN comprises exactly one population of identical TSACs which self-assemble to display emergent properties (a “single-component” TSAN). In some embodiments, an inventive TSAN comprises two or more populations of different TSACs which can assemble to display emergent properties (a “two- or multiple-component” TSAN).

The invention provides pharmaceutical compositions for delivery of inventive TSACs and/or TSANs to a subject. In some embodiments, pharmaceutical compositions of the present invention comprise inventive TSACs and/or TSANs and at least one pharmaceutically acceptable carrier.

In some embodiments, a therapeutic amount of an inventive composition is administered to a subject for therapeutic and/or diagnostic purposes. In some embodiments, the amount of TSAN and/or TSAC is sufficient to treat and/or diagnose a disease, condition, and/or disorder.

The invention provides methods and compositions by which TSACs may be triggered to self-assemble at target sites (e.g. organ, tissue, cell, and/or intracellular domain) to locally activate one or more emergent properties. In some embodiments, such locally-activated emergent properties can be used for diagnostic and/or therapeutic purposes. In some embodiments, inventive TSANs and/or TSACs may be used to diagnose and/or treat

Any disease, disorder, and/or condition may be treated using inventive TSANs and/or TSACs. In particular, any disease, disorder, and/or condition that has an inflammatory component may be treated using inventive compositions and methods. In some embodiments, inventive TSANs and/or TSACs may be used to treat a cell proliferative disorder.

The invention provides a variety of kits for conveniently and/or effectively carrying out methods of the present invention. Inventive kits comprise one or more TSANs and/or TSACs. In some embodiments, kits comprise a collection of different TSANs and/or TSACs to be used for different purposes (e.g. diagnostics and/or treatment). In some embodiments, inventive kits comprise one or more TSANs and/or TSACs of the invention. In some embodiments, such a kit is used in the diagnosis and/or treatment of a subject suffering from and/or susceptible to a disease, condition, and/or disorder (e.g. cancer). In some embodiments, the invention provides kits for identifying TSANs and/or TSACs which are useful in treating and/or diagnosing a disease, disorder, and/or condition.

### BRIEF DESCRIPTION OF THE DRAWING

FIGS. 1A-B. Schematic of inventive methods and compositions. (A) A general schematic of elements of compositions of the invention. (B) An example of proteolytic actuation. NeutrAvidin- and biotin-functionalized superparamagnetic iron-oxide TSACs are inhibited by the attachment of PEG chains that are anchored by MMP-2-cleavable peptide substrates (GPLGVRGC). Upon proteolytic removal of PEG via cleavage of the peptides, biotin and NeutrAvidin TSACs self-assemble into nanoassemblies with enhanced magnetic susceptibility, T2 magnetic resonance relaxation, and lowered diffusivity.

FIGS. 2A-D. The role of PEG length and characterization of assembly. (A) Changes in light scattering of TSACs over time with MMP-2 (11 μg/ml) (hollow) or without MMP-2 (solid) shows PEG length influence on TSAC aggregation kinetics. (B) Difference between extinction of TSACs with and without MMP-2 after 3 hours reveals PEG chain length of 10 kDa. (C) TSACs with specific MMP-2 substrate aggregate in the presence of MMP-2 (11 μg/ml) whereas TSACs with scrambled peptide do not. (D) Atomic Force Micrographs of TSAC solutions in (C) confirm aggregation of TSACs in the presence of MMP-2. Scale bars are 500 nm.

FIG. 3. MMP-2 triggered self-assembly results in detectable changes in T2 relaxation times. T2 maps generated by a 4.7T Bruker MRI shows detectable aggregation after 3 hours with the addition of 85, 170, 340, 680, and 1360 ng/ml MMP-2 for TSAC concentrations of 32 pM, 10 pM, and 3.2 pM respectively.

FIGS. 4A-C. Triggered self-assembly of TSACs by HT-1080 tumor-derived cells. (A) T2 mapping of Fe₃O₄ TSACs incubated for 5 hours over HT-1080 cells that secrete active MMP-2 in a complex medium. TSAC assembly amplifies T2 relaxation over cancer cells relative to cells incubated with the MMP inhibitor Galardin at 25 μM. (B) Activated TSACs are drawn out of solution by a strong magnet (left) while inactive TSACs (right) are not. (C) TSACs activated by MMP-2 secreting tumor cells for 3 hours are drawn out of solution onto cells by a magnetic field. Available NeutrAvidin on aggregates is stained with biotin-quantum dots (Em: 605 nm) and imaged by epifluorescent microscopy. Assemblies are not targeted to cells if an MMP inhibitor is used. Scale bar represents 50 μm.

FIG. 5. Polymer-coated, superparamagnetic TSACs were modified with either a tyrosine-containing kinase substrate or an SH2 domain. As kinases phosphorylate substrates, SH2 TSACs recognize and bind phosphopeptide TSACs, thereby coupling TSAC assembly to the presence of kinase activity. Assembly, in turn, amplifies the T2 relaxation in MRI, allowing NMR-based kinase detection. TSAC assembly is reversible through phosphatase removal of phosphate modifications.

FIG. 6. Phosphopeptide (pY) TSAC assembly with SH2 TSACs. Upon addition of SH2 TSACs to pY-peptide TSACs, rapid increase in hydrodynamic radius was observed by DLS (dark dots). In the presence of free pY-peptide, TSAC assembly was not observed (diamonds). Non-phosphorylated peptide and non-binding pY-peptide remain dispersed with SH2 TSACs, demonstrating both sequence- and phosphate-specific peptide recognition by SH2 TSACs (squares and light dots, respectively). Assembly was reversed by addition of excess free pY-peptide to the mixture after a 10 minute incubation (triangles).

FIG. 7. Kinase-directed TSAC assembly. (A) 5 U/μl Abl kinase (light dots) was added to a mixture of SH2 TSACs and tyrosine-containing, Abl substrate TSACs at 2 minutes and TSAC radius was observed over time using dynamic light scattering (DLS). Controls without kinase (dark dots) with phenylalanine-Abl substrate TSACs (triangles) did not assemble. (B) In MRI, T2 relaxation is enhanced by Abl kinase-directed, assembly (bottom two wells) and was reversed by addition of 200 μM free phosphopeptide, but not by mixing alone. Controls lacking enzyme (top), containing phenylalanine substrate TSACs (second from top), or 200 μM free pY substrate (third from top) did not show enhancement. (C) Dose-dependent T2 relaxation enhancement of SH2 TSACs and Y-peptide TSACs 3 hours following Abl kinase addition (12 nM TSACs).

FIG. 8. Phosphatase reversal of TSAC assembly in DLS and MRI. (A) SH2 TSACs and pY-Abl substrate TSACs were allowed to assemble prior to addition of 2 U/μl phosphatase (red) or vehicle control (blue) at 25 minutes. (B) TSACs were exposed to 2.5 U/μl Abl kinase followed by 5 U/μl phosphatase. (C) Kinase-directed assembly and phosphatase disassembly was visualized via T2 relaxation enhancement in MRI.

FIG. 9. Schematic representation of logical TSAC sensors. Self-assembly is gated to occur in the presence of MMP-2 and MMP-7 (Logical “AND,” Left) or in the presence of either or both proteases (Logical “OR,” Right) by attachment of protease-removable polyethylene glycol polymers to complementary TSACs.

FIG. 10. Logical “AND.” (A) Hydrodynamic radius in dynamic light scattering is increased only in the presence of both MMP-2 and MMP-7. Either or none is insufficient to actuate assembly (40 μg Fe/ml). (B) Assemblies express “AND” logic in MRI. T2 relaxation decreases approximately 30% in 3 hours following addition of 0.2 μg MMP-2 and 0.2 μg MMP-7, with nominal changes following addition of either enzyme alone (7.5 μg Fe/ml).

FIG. 11. Logical “OR.” (A) Population hydrodynamic radius is increased in the presence of either or both MMP-2 and MMP-7 (40 μg/ml Fe). (B) MRI visualization of logical function demonstrates approximately 40% enhancement in T2 relaxation in the presence of either 0.4 μg MMP-2 or 0.2 μg MMP-7 or both enzymes (0.2 μg MMP-2 and 0.1 μg MMP-7) (15 μg/ml Fe).

FIG. 12. Probing TSAC latency and specificity using dynamic light scattering. (A) Ligand-TSACs were masked with MMP-2-PEG to inhibit assembly with unmodified receptor TSACs (40 μg Fe/ml). Addition of 0.4 μg MMP-2 actuates TSAC assembly, while 0.4 μg MMP-7 or no enzyme is insufficient. (B) Receptor-TSACs were masked with MMP-7-PEG to inhibit assembly with unmodified ligand TSACs (40 μg Fe/ml). Here, addition of 0.4 μg MMP-7 induces assembly, while 0.4 μg MMP-2 cannot.

### DEFINITIONS

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, and/or worms. In some embodiments, an animal may be a transgenic animal, genetically-engineered animal, and/or a clone.

Approximately: As used herein, the terms “approximately” or “about” in reference to a number are generally taken to include numbers that fall within a range of 10% in either direction (greater than or less than) of the number unless otherwise stated or otherwise evident from the context (except where such number would exceed 100% of a possible value).

Blocking agent: As used herein, the term “blocking agent” refers to agents which mask, block, cloak, and/or sterically inhibit the activity, self-recognition, and/or self-assembly of complementary binding moieties. Inventive triggered self-assembly conjugates (TSACs) may comprise a blocking agent which blocks the ability of complementary binding moieties to interact with one another prior to a desired condition or time. In specific embodiments, the presence of a blocking agent on the surface of a TSAC sterically inhibits self-assembly until removal of the blocking agent by cleavage of the cleavable substrate. Examples of blocking agents include, but are not limited to, polaxamines, poloxamers, polyethylene glycol (PEG), peptides, or other synthetic polymers of sufficient length and density to both mask self-assembly and provide protection against non-specific adsorption, opsonization, and reticuloendothelial system (RES) uptake.

Cargo domain: As used herein, the term “cargo domain” refers to a region or portion of a cargo entity, such that each region or portion has little to no desired effect by itself, but when combined have an increased effect. By “complementary cargo domain” is meant that a first cargo domain complements a second cargo domain to become “activated.” Exemplary cargo domains include, but are not limited to, fluorescent moieties, quantum dots, molecular beacons, organic fluorophores, bioluminescent proteins (e.g., luciferase), etc.

Cargo entity: As used herein, the term “cargo entity” refers to any substance that is capable of conjugation to a monomeric unit of a triggered self-assembly conjugate (TSAC). In some embodiments, a cargo entity is a substance that, by itself, has little to no desired effect; however, upon self-assembly (e.g., upon interaction of TSAC complementary binding moieties), cargo entities can interact to achieve a desired result (e.g. magnetic, optical, or fluorescent properties). In some embodiments, a cargo entity is a molecule, material, substance, and/or construct that can be delivered to a cell by conjugation to a TSAC and/or TSAN. Cargo entities may comprise one or more cargo domains, which are defined herein. As used herein, the term “cargo entity” is interchangeable with “payload.”

Cleavable linker: As used herein, the term “cleavable linker” refers to a moiety by which a blocking agent is conjugated to a complementary binding moiety or to a monomeric unit of a TSAC. In general, cleavage of the cleavable linker allows for removal of the blocking agent, which permits TSAC self-assembly. Cleavable linkers of the invention may be cleaved via any form of cleavable chemistry. Exemplary cleavable linkers include, but are not limited to, protease cleavable peptide linkers, nuclease sensitive nucleic acid linkers, lipase sensitive lipid linkers, glycosidase sensitive carbohydrate linkers, pH sensitive linkers, hypoxia sensitive linkers, photo-cleavable linkers, heat-labile linkers, enzyme cleavable linkers, ultrasound-sensitive linkers, x-ray cleavable linkers, etc.

Complementary binding moiety: As used herein, the term “complementary binding moiety” refers to sets of molecules, substances, moieties, entities, and/or agents that are capable of self-recognition and association. Complementary binding moieties are typically conjugated to monomeric units within inventive TSACs. One of ordinary skill in the art will appreciate that any complementary binding moiety can be used in accordance with the present invention. Exemplary complementary binding moieties include, but are not limited to, ligands and anti-ligands (e.g. streptavidin and biotin), ligands and receptors (e.g. small molecule ligands and their receptors), antibodies and antigens, phage display-derived peptides, complementary nucleic acids (e.g. DNA hybrids, RNA hybrids, DNA/RNA hybrids, etc.), and aptamers. In some embodiments, complementary binding moieties include streptavidin and biotin. Other exemplary complementary binding moieties include, but are not limited to, moieties exhibiting complementary charges, hydrophobicity, hydrogen bonding, covalent bonding, Van der Waals forces, reactive chemistries, electrostatic interactions, magnetic interactions, etc.

Conjugated. As used herein, the terms “conjugated,” “linked,” “attached,” and “associated with,” when used with respect to two or more moieties, means that the moieties are physically associated or connected with one another, either directly or via one or more additional moieties that serves as a linking agent, to form a structure that is sufficiently stable so that the moieties remain physically associated under the conditions in which structure is used, e.g., physiological conditions. Typically the moieties are attached either by one or more covalent bonds or by a mechanism that involves specific binding. Alternately, a sufficient number of weaker interactions can provide sufficient stability for moieties to remain physically associated.

Diagnostic agent: As used herein, the term “diagnostic agent” refers to refers to any agent that, when administered to a subject, facilitates the diagnosis of a disease, disorder, and/or condition.

Emergent property: As used herein, the term “emergent property” refers to any property which exists when two entities, substances, and/or moieties are brought together, associated, and/or conjugated, but does not exist when the entities, substances, and/or moieties are separate. Emergent properties may be electrical, magnetic, optical, mechanical, and/or biological. In some embodiments, emergent properties can be assayed and/or measured. For the purposes of the present invention, an emergent property is one that is exhibited by triggered self-assembly conjugate (TSAC) aggregates, but is not exhibited by individual TSACs that have not undergone self-assembly.

In vitro: As used herein, the term “in vitro” refers to events that occur in an artificial environment, e.g., in a test tube or reaction vessel, in cell culture, etc., rather than within an organism (e.g. animal, plant, and/or microbe).

In vivo: As used herein, the term “in vivo” refers to events that occur within an organism (e.g. animal, plant, and/or microbe).

Monomeric unit: As used herein, the term “monomeric unit” refers to any substance capable of conjugation to a complementary binding moiety. In general, a monomeric unit is a component of a triggered self-assembly conjugate (TSAC). One of ordinary skill in the art will appreciate that any monomeric unit can be used in inventive TSACs. To give but a few examples, a monomeric unit may be a nanoparticle, microparticle, dendrimer, nanoemulsion, liposome, polymer, micelle, protein, peptide, etc. In certain embodiments, a monomeric unit is a nanoparticle. In certain embodiments, a monomeric unit is a microparticle.

Nanoparticle: As used herein, the term “nanoparticle” refers to any particle having a diameter of less than 1000 nanometers (nm). In some embodiments, nanoparticles can be optically or magnetically detectable. In some embodiments, intrinsically fluorescent or luminescent nanoparticles, nanoparticles that comprise fluorescent or luminescent moieties, plasmon resonant nanoparticles, and magnetic nanoparticles are among the detectable nanoparticles that are used in various embodiments of the invention. In general, the nanoparticles should have dimensions small enough to allow their uptake by eukaryotic cells. Typically the nanoparticles have a longest straight dimension (e.g., diameter) of 200 nm or less. In some embodiments, the nanoparticles have a diameter of 100 nm or less. Smaller nanoparticles, e.g., having diameters of 50 nm or less, e.g., 5-30 nm, are used in some embodiments of the invention. In certain embodiments of the invention, the nanoparticles are quantum dots, i.e., bright, fluorescent nanocrystals with physical dimensions small enough such that the effect of quantum confinement gives rise to unique optical and electronic properties. In certain embodiments of the invention, the optically detectable nanoparticles are metal nanoparticles. Metals of use in the nanoparticles include, but are not limited to, gold, silver, iron, cobalt, zinc, cadmium, nickel, gadolinium, chromium, copper, manganese, palladium, tin, and alloys and/or oxides thereof. In some embodiments, magnetic nanoparticles are of use in the invention. “Magnetic particles” refers to magnetically responsive particles that contain one or more metals or oxides or hydroxides thereof.

Self-assembly: As used herein, the term “self-assembly” refers to a process of spontaneous assembly of a higher order structure that relies on the natural attraction of the components of the higher order structure (e.g., molecules) for each other. It typically occurs through random movements of the molecules and formation of bonds based on size, shape, composition, or chemical properties.

Small molecule: In general, a “small molecule” is understood in the art to be an organic molecule that is less than about 5 kilodaltons (Kd) in size. In some embodiments, the small molecule is less than about 3 Kd, 2 Kd, or 1 Kd. In some embodiments, the small molecule is less than about 800 daltons (D), 600 D, 500 D, 400 D, 300 D, 200 D, or 100 D. In some embodiments, small molecules are non-polymeric. In some embodiments, small molecules are not proteins, peptides, or amino acids. In some embodiments, small molecules are not nucleic acids or nucleotides. In some embodiments, small molecules are not saccharides or polysaccharides.

Specific binding: As used herein, the term “specific binding” refers to non-covalent physical association of a first and a second moiety wherein the association between the first and second moieties is at least 100 times as strong as the association of either moiety with most or all other moieties present in the environment in which binding occurs. Binding of two or more entities may be considered specific if the equilibrium dissociation constant, K_{d}, is 10⁻⁶ M or less, 10⁻⁷ M or less, 10⁻⁸ M or less, or 10⁻⁹ M or less under the conditions employed, e.g., under physiological conditions such as those inside a cell or consistent with cell survival. Examples of specific binding interactions include antibody-antigen interactions, avidin-biotin interactions, hybridization between complementary nucleic acids, etc.

Subject: As used herein, the term “subject” or “patient” refers to any organism to which a composition of this invention may be administered, e.g., for experimental, diagnostic, and/or therapeutic purposes. Typical subjects include animals (e.g., mammals such as mice, rats, rabbits, non-human primates, and humans) and/or plants.

Suffering from: An individual who is “suffering from” a disease, disorder, and/or condition has been diagnosed with or displays one or more symptoms of a the disease, disorder, and/or condition.

Therapeutically effective amount: As used herein, the term “therapeutically effective amount” means an amount of a therapeutic and/or diagnostic agent (e.g., TSAN, TSAC) that is sufficient, when administered to a subject suffering from or susceptible to a disease, disorder, and/or condition, to treat and/or diagnose the disease, disorder, and/or condition.

Therapeutic agent: As used herein, the phrase “therapeutic agent” refers to any agent that, when administered to a subject, has a therapeutic effect and/or elicits a desired biological and/or pharmacological effect.

Treating: As used herein, the term “treating” refers to partially or completely alleviating, ameliorating, relieving, delaying onset of, inhibiting progression of, reducing severity of, and/or reducing incidence of one or more symptoms or features of a particular disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition. In some embodiments, treatment comprises delivery of a TSAN and/or TSAC to a subject.

Triggered self-assembly conjugate (TSAC): As used herein, the term “triggered self-assembly conjugate,” or “TSAC” refers to any composition that aggregates and/or self-assembles upon activation by a trigger. In general, TSACs comprise one or multiple monomeric units and one or more complementary binding moieties. In general, monomeric units are conjugated to complementary binding moieties, which can mediate triggered self assembly. In some embodiments, TSAC aggregates formed by triggered self-assembly display electrical, magnetic, optical, mechanical, and/or biological properties (i.e. emergent properties) which are not displayed by individual TSACs. Exemplary triggers include, but are not limited to, proteins (e.g. enzymes), nucleic acids (e.g. RNase, ribozyme, DNase), light, x-rays, ultrasound radiation, pH, heat, hypoxic conditions, etc. Inventive TSACs may optionally comprise a blocking agent which prevents complementary binding moieties from being able to interact and promote self-assembly. Combinations of TSAC populations can serve as triggered self-assembly nanosystems (TSANs).

Triggered self-assembly nanosystem (TSAN): As used herein, the term “triggered self-assembly nanosystem,” or “TSAN” refers to a nanosystem characterized by populations of individual components that are able to aggregate and/or self-assemble upon activation by a trigger. In some embodiments, the individual components may be triggered self-assembly conjugates (TSACs). In some embodiments, any trigger may be used to activate self-assembly of individual components (e.g. TSACs). Exemplary triggers include, but are not limited to, proteins (e.g. enzymes), nucleic acids (e.g. RNase, ribozyme, DNase), light, x-rays, ultrasound radiation, pH, heat, hypoxic conditions, etc.

### DETAILED DESCRIPTION OF CERTAIN EMBODIMENTS

### Triggered Self-Assembly Nanosystem (TSAN)

The present invention provides triggered self-assembly nanosystems (TSAN). In general, TSANs comprise individual components (i.e. triggered self-assembly conjugates (TSACs), described herein) that are able to aggregate or self-assemble upon activation by a trigger.

Any trigger can be used to activate self-assembly of individual TSACs. To give but a few examples, triggers can be proteins (e.g. enzymes), nucleic acids (e.g. RNase, ribozyme, DNase), light, x-rays, ultrasound radiation, pH, heat, hypoxic conditions, etc.

In certain embodiments, a trigger is or comprises an enzyme (e.g. lipase, glycosidase, protease, DNAse, RNAse, etc.). In some embodiments, a trigger is or comprises an enzyme that recognizes a specific peptide sequence and/or peptide structure. In some embodiments, a trigger is or comprises an enzyme that recognizes a specific nucleic acid sequence and/or structure. In some embodiments, a trigger is or comprises an enzyme that recognizes a specific carbohydrate composition and/or structure. In some embodiments, a trigger is or comprises an enzyme that recognizes a specific lipid composition and/or structure.

In some embodiments, a trigger is or comprises an enzyme that catalyzes cleavage of a peptide. In some embodiments, a trigger is or comprises an enzyme that catalyzes cleavage of a nucleic acid. In some embodiments, a trigger is or comprises an enzyme that catalyzes cleavage of a carbohydrate. In some embodiments, a trigger is or comprises an enzyme that catalyzes cleavage of a lipid. In some embodiments, a trigger is or comprises an enzyme that recognizes a specific peptide, nucleic acid, carbohydrate, and/or lipid sequence, composition, and/or structure and catalyzes cleavage of the peptide, nucleic acid, carbohydrate, and/or lipid.

In some embodiments, a trigger is or comprises an enzyme that modifies a peptide. In some embodiments, a trigger is or comprises an enzyme that modifies a nucleic acid. In some embodiments, a trigger is or comprises an enzyme that modifies a carbohydrate. In some embodiments, a trigger is or comprises an enzyme that modifies a lipid.

In some embodiments, an enzyme trigger may modify the structure of a peptide, nucleic acid, carbohydrate, and/or lipid (e.g., by the formation of cross-links). In some embodiments, an enzyme trigger may modify the shape of the peptide, nucleic acid, carbohydrate, and/or lipid.

In some embodiments, an enzyme trigger may modify the charge of one or more charged surface groups of a peptide, nucleic acid, carbohydrate, and/or lipid. In some embodiments, an enzyme trigger may modify a peptide, nucleic acid, carbohydrate, and/or lipid by removing one or more surface groups (e.g. phosphate, acetyl, methyl, maleimide, etc.) to the peptide, nucleic acid, carbohydrate, and/or lipid. In some embodiments, an enzyme trigger may modify a peptide, nucleic acid, carbohydrate, and/or lipid by adding one or more surface groups (e.g. phosphate, acetyl, methyl, maleimide, etc.) to the peptide, nucleic acid, carbohydrate, and/or lipid. In some embodiments, an enzyme trigger may modify one or more surface groups (e.g. phosphate, acetyl, methyl, maleimide, etc.) of a peptide, nucleic acid, carbohydrate, and/or lipid. To give but one example, a trigger may be an enzyme (e.g. a kinase) that attaches a surface group (e.g. a phosphate group) to a peptide.

In certain embodiments, a trigger is or comprises a nucleic acid. In certain specific embodiments, a trigger is or comprises an RNase (e.g. RNase A, RNase H, RNase III, RNase T1, RNase T2, RNase U2, RNase V1, RNase I, RNase L, RNase PhyM, RNase V, etc.). In certain specific embodiments, a trigger is or comprises a ribozyme. In certain specific embodiments, a trigger is or comprises a DNase (e.g. DNase I, DNase II alpha, DNase II beta, etc.). Such nucleic acid triggers can be useful for acting upon a cleavable linker comprising a nucleic acid sequence. To give but one example, a blocking agent may be conjugated to a TSAC via a cleavable linker comprising RNA. In the presence of RNase, the linker is cleaved and TSACs are allowed to self-assemble.

In certain embodiments, a trigger is or comprises light. In some embodiments, light may facilitate hydrolysis, degradation, and/or cleavage of a chemical bond associated with a peptide, nucleic acid, carbohydrate, and/or lipid.

In certain embodiments, a trigger is or comprises an x-ray. In some embodiments, an x-ray trigger can cleave a chemical bond directly. In some embodiments, an x-ray trigger can cleave a chemical bond through an interaction with the TSAC core.

In certain embodiments, a trigger is or comprises a condition characterized by a particular pH. In certain embodiments, a trigger is or comprises a condition characterized by a change in pH. In some embodiments, pH may facilitate hydrolysis, degradation, and/or cleavage of a chemical bond associated with a peptide, nucleic acid, carbohydrate, and/or lipid. In some embodiments, pH may modify the charge and/or electrostatic force of a peptide, nucleic acid, carbohydrate, and/or lipid. In some embodiments, pH may rearrange surface packing of a peptide, nucleic acid, carbohydrate, and/or lipid. In some embodiments, pH may alter secondary structures of a peptide, nucleic acid, carbohydrate, and/or lipid. To give but one example, conditions characterized by high pH may promote the formation of inter- and intra-molecular disulfide bonds (e.g. a bond formed between any two cysteine residues) to a greater extent than conditions characterized by low pH.

In certain embodiments, a trigger is or comprises a condition characterized by heat. In some embodiments, heat may facilitate hydrolysis, degradation, and/or cleavage of a chemical bond associated with a peptide, nucleic acid, carbohydrate, and/or lipid. In some embodiments, heat may alter inter- and intra-molecular hydrogen bonding associated with a peptide, nucleic acid, carbohydrate, and/or lipid. In some embodiments, heat may modify a peptide, nucleic acid, carbohydrate, and/or lipid by initiating phase changes.

In certain embodiments, a trigger is a or comprises condition characterized by hypoxia. In certain embodiments, hypoxia leads to conditions characterized by the presence of singlet oxygen and/or radical oxygen species. In some embodiments, singlet oxygen and/or radical oxygen species may facilitate hydrolysis, degradation, and/or cleavage of a chemical bond associated with a peptide, nucleic acid, carbohydrate, and/or lipid. In some embodiments, singlet oxygen and/or radical oxygen species may modify a peptide, nucleic acid, carbohydrate, and/or lipid by removing one or more surface groups (e.g. phosphate, acetyl, methyl, maleimide, etc.) to the peptide, nucleic acid, carbohydrate, and/or lipid. In some embodiments, singlet oxygen and/or radical oxygen species may modify a peptide, nucleic acid, carbohydrate, and/or lipid by adding one or more surface groups (e.g. phosphate, acetyl, methyl, maleimide, etc.) to the peptide, nucleic acid, carbohydrate, and/or lipid. In some embodiments, singlet oxygen and/or radical oxygen species may modify one or more surface groups (e.g. phosphate, acetyl, methyl, maleimide, etc.) of a peptide, nucleic acid, carbohydrate, and/or lipid.

### Emergent Properties

In some embodiments, an “emergent property” refers to a shift, enhancement, and/or reduction of plasmon resonance that depends on the assembly of TSACs into aggregates. Such enhanced properties can be used for imaging or activation/excitation. In some embodiments, coupling of plasmons from two or more assembled nanoparticles (e.g. TSACs) provides a stronger or shifted resonance peak that can be distinguished from a resonance peak of a single nanoparticle (e.g. TSAC). An altered plasmon resonance peak could be excited and/or detected with a laser and/or light source specific for the wavelength of the peak.

Emergent Electrical Properties

In some embodiments, an “emergent property” refers to a shift, enhancement, and/or reduction of electrical resonance that depends on the assembly of TSACs into aggregates. Such enhanced properties can be used for imaging or activation/excitation.

Plasmon resonance is an electrical property of a material that has been excited by electromagnetic (EM) energy at light wavelengths. Plasmon resonance allows for coupling of significant energy to nanomaterials (e.g. TSACs). Metal nanoparticles that differ in size and composition tend to scatter light of different wavelengths according to their distinct surface plasmon resonances, and these differences can be measured and analyzed.

Briefly, when an external electro-magnetic field such as light is applied to a metal, conduction electrons move collectively so as to screen the perturbed charge distribution, in what is known as “plasma oscillation.” Surface plasmon resonance (SPR) is, hence, a collective excitation mode of plasma localized near a metal surface.

In the case of a metal nanoparticle, surface plasmon mode is “restricted” due to the small dimensions to which electrons are confined, i.e., surface plasmon mode must conform to the boundaries of the dimensions of the nanoparticle. Therefore, the resonance frequency of the surface plasmon oscillation of the metal nanoparticle is different from the plasma frequency of the bulk metal. Surface interactions can alter optical properties and influence the spectral profile of the light scattered by the SPR of the metal nanoparticles. This feature can be applied as an indicator in sensing interactions.

To give but one example, gold and silver nanoparticles are commonly used for measuring plasmon resonance. In typical biosensors based on gold nanoparticles, the color change which may be observed is usually caused by aggregation. Aggregation of individual gold nanoparticles gives rise to a color change. In general, a decrease in absorbance (usually measured at 260 nm) and a broadening of the spectra generated by plasmon resonance analysis may be attributed to aggregation of gold nanoparticles. Individual gold nanoparticles appear crimson in color to the naked eye, but larger aggregates of gold nanoparticles appear blue.

In some embodiments, near infrared (NIR) lasers coupled to a shifted or enhanced plasmon peak can be used to generate heat from the excited plasmon. Heat can be used to destroy tissue, activate/release a diagnostic and/or therapeutic agent. Heat can also modify tissue architecture for subsequent diagnostics, targeting, imaging, therapeutics, etc.

In some embodiments, carbon nanotubes; quantum dots; and/or gold, silver, and/or other conductive and/or semiconductive nanorods and/or nanoparticles (e.g. TSACs) can be assembled into electronic circuits that can perform electrochemistry, sensing, communication, computing, etc. In some embodiments, such self-assembled circuits approach micro-scale dimensions and communicate through longer-wavelength EM, e.g., radio frequency (RF).

Emergent Magnetic Properties

In some embodiments, an “emergent property” refers to a shift, enhancement, and/or reduction of magnetic resonance that depends on the assembly of TSACs into aggregates. Such altered properties can be used for imaging or activation/excitation. In some embodiments, emergent properties result from magnetic nanoparticles which assemble their dipoles coordinately to form a net dipole that is greater than the sum of the parts.

In some embodiments, measurement and/or detection of emergent properties can be used for enhanced MRI imaging, magnetic nanoparticle imaging, and/or other modalities that utilize strength of magnetic dipole for contrast. In some embodiments, self-assembly of TSACs can activate a detection signal, such as the decreased T2 weighted signal in MRI of closely associated iron-oxide nanoparticles (e.g. TSACs). There also exists the potential for multiplexing sensors by encoding target specificity into the formation of assemblies with unique combinations of electromagnetic nanoparticles.

Detection of emergent magnetic properties may be performed using any method known in the art. For example, a magnetometer or a detector based on the phenomenon of nuclear magnetic resonance (NMR) can be employed. Superconducting quantum interference devices (SQUID), which use the properties of electron-pair wave coherence and Josephson junctions to detect very small magnetic fields can be used. Magnetic force microscopy or handheld magnetic readers can be used. U.S Patent Publication 2003/009029 describes various suitable methods. Magnetic resonance microscopy offers one approach (Wind et al., 2000, J. Magn. Reson., 147:371).

Emergent magnetic properties can be detected and/or measured by analyzing T2 relaxation times using magnetic resonance imaging (MRI), magnetic field manipulation, etc. In general, MRI is based upon the relaxation properties of excited hydrogen nuclei. Briefly, all nuclei that contain odd numbers of protons or neutrons have an intrinsic magnetic moment and angular momentum. Magnetic nuclei are aligned with a strong external magnetic field, and the alignment is disturbed using an electromagnetic field that is perpendicular to the external magnetic field. The resulting response to the perturbing electromagnetic field is exploited in MRI, providing detailed information regarding topology, dynamics, and three-dimensional structure of molecules and nanoparticle aggregates. Nanoassemblies (e.g. TSAC aggregates) typically display shorter T2 relaxation times as measured by MRI relative to individual nanoparticles (e.g. individual TSACs).

Magnetic field manipulation generally exploits the relative behaviors of nanoparticle aggregates versus individual nanoparticles in the presence of a magnet. Briefly, as magnetic domains of aggregated nanoparticles (e.g. TSACs) coordinate to form an amplified cumulative dipole, they become more susceptible to long-range dipolar forces. This phenomenon allows manipulation of nanoassemblies (e.g. TSAC aggregates) with imposed magnetic fields, while isolated nanoparticles (e.g. individual TSACs) remain unaffected. Typically, aggregates of nanoparticles can be distinguished from individual nanoparticles because aggregates can be visually drawn out of solution by a strong magnet while individual nanoparticles remain disperse.

Emergent Optical Properties

In some embodiments, an “emergent property” refers to a shift, enhancement, and/or reduction of optical resonance that depends on the assembly of TSACs into aggregates. Such enhanced properties can be used for imaging or activation/excitation. For example, assembly of gold nanoparticles changes the plasmon resonance of individual gold nanoparticles which can lead to changes in light scattering and absorbance. To give another example, self-assembly of individual TSACs into TSAC aggregates enhances the light scattering properties of the TSAC as contributions of Mie scattering emerge. The absorbtion/scattering cross-section broadens with assembly, potentially amplifying the sensitivity of detection. Such emergent optical properties can be used for optical detection with spectroscopy, optical coherence tomography (OCT), reflectance imaging, and/or other optical techniques or for excitation with resultant heating.

Detection of emergent optical properties is accomplished by detecting scattering, emission, and/or absorption of light that falls within the optical region of the spectrum, i.e., that portion of the spectrum extending from approximately 180 nm to several microns. Optionally a sample containing cells is exposed to a source of electromagnetic energy. In some embodiments of the invention, absorption of electromagnetic energy (e.g., light of a given wavelength) by a nanoparticle or a component thereof is followed by emission of light at longer wavelengths, and the emitted light is detected. In some embodiments, scattering of light by nanoparticles is detected. In certain embodiments of the invention, light falling within the visible portion of the electromagnetic spectrum, i.e., the portion of the spectrum that is detectable by the human eye (approximately 400 nm to approximately 700 nm) is detected. In some embodiments of the invention, light that falls within the infrared or ultraviolet region of the spectrum is detected.

A detectable emergent optical property can be a feature of an absorption, emission, or scattering spectrum or a change in a feature of an absorption, emission, or scattering spectrum. A detectable emergent optical property can be a visually detectable feature such as, for example, color, apparent size, or visibility (i.e. simply whether or not the nanoparticle is visible under particular conditions). Features of a spectrum include, for example, peak wavelength or frequency (wavelength or frequency at which maximum emission, scattering intensity, extinction, absorption, etc. occurs), peak magnitude (e.g., peak emission value, peak scattering intensity, peak absorbance value, etc.), peak width at half height, or metrics derived from any of the foregoing such as ratio of peak magnitude to peak width. Certain spectra may contain multiple peaks, of which one is typically the major peak and has significantly greater intensity than the others. Each spectral peak has associated features. Typically, for any particular spectrum, spectral features such as peak wavelength or frequency, peak magnitude, peak width at half height, etc., are determined with reference to the major peak. The features of each peak, number of peaks, separation between peaks, etc., can be considered to be features of the spectrum as a whole. The foregoing features can be measured as a function of the direction of polarization of light illuminating the nanoparticles; thus polarization dependence can be measured. Features associated with hyper-Rayleigh scattering can be measured.

In some embodiments, emergent optical properties can be measured using optical tomography, for example, optical coherence tomography (OCT). In general, optical tomography creates a digital volumetric model of an object by reconstructing images made from light transmitted and scattered through an object; thus, optical tomography relies on the object under study being at least partially light-transmitting. Optical tomography most commonly used for medical imaging.

In some embodiments, emergent optical properties can be emergent fluorescent properties. For example, fluorescent particles (e.g. quantum dots), when assembled with gold nanoparticles, may undergo quenching (i.e., fluorescence reduction) or fluorescence enhancement, depending on the structure of the assembly.

Emergent fluorescent or luminescent properties can be detected using any approach known in the art including, but not limited to, spectrometry, fluorescence microscopy, flow cytometry, etc. Spectrofluorometers and microplate readers are typically used to measure average properties of a sample while fluorescence microscopes resolve fluorescence as a function of spatial coordinates in two or three dimensions for microscopic objects (e.g., less than approximately 0.1 mm diameter). Microscope-based systems are thus suitable for detecting and optionally quantitating nanoparticles inside individual cells.

Flow cytometry measures properties such as light scattering and/or fluorescence on individual cells in a flowing stream, allowing subpopulations within a sample to be identified, analyzed, and optionally quantitated (see, e.g., Mattheakis et al., 2004, Analytical Biochemistry, 327:200; Chattopadhyay et al., 2006). Multiparameter flow cytometers are available. In certain embodiments of the invention, laser scanning cytometery is used (77). Laser scanning cytometry can provide equivalent data to a flow cytometer but is typically applied to cells on a solid support such as a slide. It allows light scatter and fluorescence measurements and records the position of each measurement. Cells of interest may be re-located, visualized, stained, analyzed, and/or photographed. Laser scanning cytometers are available, e.g., from CompuCyte (Cambridge, Mass.).

In certain embodiments of the invention, an imaging system comprising an epifluorescence microscope equipped with a laser (e.g., a 488 nm argon laser) for excitation and appropriate emission filter(s) is used. The filters should allow discrimination between different populations of nanoparticles used in the particular assay. For example, in one embodiment, the microscope is equipped with fifteen 10 nm bandpass filters spaced to cover portion of the spectrum between 520 and 660 nm, which would allow the detection of a wide variety of different fluorescent particles. Fluorescence spectra can be obtained from populations of nanoparticles using a standard UV/visible spectrometer.

Emergent Mechanical Properties

In some embodiments, an “emergent property” refers to a change in mechanical properties that depends on the assembly of TSACs into aggregates. Just as short collagen fragments can form gels with macroscopic mechanical properties, or as blood proteins can clot to form a new tissue, TSAC aggregates provide novel mechanical properties that may enhance their biological efficacy. To give but one example, TSAC aggregates may have altered mechanical properties (e.g. enhanced strength and support) relative to individual TSACs.

Emergent Biological Properties

In some embodiments, an “emergent property” refers to a change in biological properties that depends on the assembly of TSACs into aggregates. Any biological property or phenomenon that is able to be detected, assayed, and/or measured can be an emergent biological property of the invention. For example, self-assembly of TSACs conjugated to biological molecules might result in activation of the biological molecule (e.g. protein, nucleic acid, carbohydrate, lipid, small molecule, drug, therapeutic agent, etc.).

In some embodiments, the biological molecule becomes active upon TSAC self-assembly. In some embodiments, the biological molecule changes its three-dimensional structure upon TSAC self-assembly. In some embodiments, the biological molecule is cleaved upon TSAC self-assembly. In some embodiments, the biological molecule is modified upon TSAC self-assembly. Such modification can include the addition or deletion of phosphate groups, methyl groups, myristoyl groups, glycosyl groups, etc. In some embodiments, the biological molecule is made more or less stable upon TSAC self-assembly. In some embodiments, the biological molecule acquires a function upon TSAC self-assembly which it does not have prior to TSAC self-assembly.

For example, a TSAN might comprise two types of TSACs: a first TSAC which comprises a protease that digests a protein of the extracellular matrix surrounding a tumor, and a second TSAC which comprises an kinase that activates the protease of the first TSAC via phosphorylation. Prior to assembly, neither TSAC on its own can perform the desired function: the protease of the first TSAC is not active until it is phosphorylated by the kinase of the second TSAC. Upon self-assembly, the TSAC aggregate brings the two enzymes together. The kinase of the second TSAC phosphorylates and activates the protease of the first TSAC, and the phosphorylated protease is now able to digest the protein of the extracellular matrix surrounding the tumor.

### Monomeric Units

The present invention provides inventive TSANs comprising individual triggered self-assembly conjugates (TSACs) that are able to aggregate or self-assemble upon activation by a trigger. In some embodiments, individual TSACs comprise one or multiple monomeric units and one or more complementary binding moieties. In general, monomeric units are conjugated to complementary binding moieties, which can mediate triggered self assembly.

One of ordinary skill in the art will appreciate that any monomeric unit can be used in accordance with the present invention. To give but a few examples, a monomeric unit may be a nanoparticle, microparticle, dendrimer, nanoemulsion, liposome, polymer, micelle, protein, peptide, etc. In certain embodiments, a monomeric unit is a nanoparticle. In certain embodiments, a monomeric unit is a microparticle.

Nanoparticles

In some embodiments, the term “nanoparticle” encompasses atomic clusters, which have a typical diameter of 1 nm or less and generally contain from several (e.g., 3-4) to up to several hundred atoms. In some embodiments, nanoparticles larger than 5 nm may reduce clearance by the kidney. In some embodiments, nanoparticles under 100 nm may be easily endocytosed in the reticuloendothelial system (RES). In some embodiments, nanoparticles under 400 nm may be characterized by enhanced accumulation in tumors. While not wishing to be bound by any theory, enhanced accumulation in tumors may be caused by the increased permeability of angiogenic tumor vasculature relative to normal vasculature. Nanoparticles can diffuse through such “leaky” vasculature, resulting in accumulation of nanoparticles in tumors.

Nanoparticles can have a variety of different shapes including spheres, oblate spheroids, cylinders, shells, cubes, pyramids, rods (e.g., cylinders or elongated structures having a square or rectangular cross-section), tetrapods (particles having four leg-like appendages), triangles, prisms, etc.

Nanoparticles can be solid or hollow and can comprise one or more layers (e.g., nanoshells, nanorings, etc.). Nanoparticles may have a core/shell structure, wherein the core(s) and shell(s) can be made of different materials. Nanoparticles may comprise gradient or homogeneous alloys: Nanoparticles may be composite particles made of two or more materials, of which one, more than one, or all of the materials possesses an electrically, magnetically, and/or optically detectable property.

It is often desirable to use a population of nanoparticles that is relatively uniform in terms of size, shape, and/or composition so that each nanoparticle has similar properties (e.g. similar electrical, magnetic, and/or optical properties). For example, at least 80%, at least 90%, or at least 95% of the nanoparticles may have a diameter or longest straight line dimension that falls within 5%, 10%, or 20% of the average diameter or longest straight line dimension.

Nanoparticles comprising one or more electrically, magnetically, and/or optically detectable materials may have a coating layer. Use of a biocompatible coating layer can be advantageous, e.g., if the nanoparticles contain materials that are toxic to cells. Suitable coating materials include, but are not limited to, proteins such as bovine serum albumin (BSA), polyethylene glycol (PEG) or a PEG derivative, phospholipid-(PEG), silica, lipids, carbohydrates such as dextran, etc. Coatings may be applied or assembled in a variety of ways such as by dipping, using a layer-by-layer technique, etc.

A variety of different nanoparticles are of use in the invention. Intrinsically fluorescent or luminescent nanoparticles, nanoparticles that comprise fluorescent or luminescent moieties, plasmon resonant nanoparticles, and magnetic nanoparticles are among the detectable nanoparticles that are used in various embodiments of the invention. In general, nanoparticles have detectable electrical, magnetic, and/or optical properties, though nanoparticles that may be detected by other approaches may be used.

An optically detectable nanoparticle is one that can be detected within a living cell using optical means compatible with cell viability. In certain embodiments of the invention, optically detectable nanoparticles are metal nanoparticles. Metals of use in the nanoparticles include, but are not limited to, gold, silver, iron, cobalt, zinc, cadmium, nickel, gadolinium, chromium, copper, manganese, palladium, tin, and alloys thereof. Oxides of any of these metals can be used.

Certain lanthanide ion-doped nanoparticles exhibit strong fluorescence and are of use in certain embodiments of the invention. A variety of different dopant molecules can be used. For example, fluorescent europium-doped yttrium vanadate (YVO₄) nanoparticles have been produced (Beaureparie et al., 2004, Nano Letters, 4:2079). Such nanoparticles may be synthesized in water and are readily functionalized with biomolecules.

Noble metals (e.g., gold, silver, copper, platinum, palladium) are typically used for plasmon resonant particles, which are discussed in further detail below. For example, gold, silver, or an alloy comprising gold, silver, and optionally one or more other metals can be used. Core/shell particles (e.g., having a silver core with an outer shell of gold, or vice versa) can be used. Particles containing a metal core and a nonmetallic inorganic or organic outer shell, or vice versa, can be used. In certain embodiments, the nonmetallic core or shell comprises or consists of a dielectric material such as silica. Composite particles in which a plurality of metal particles are embedded or trapped in a nonmetal (e.g. a polymer or a silica shell) may be used. Hollow metal particles (e.g., hollow nanoshells) having an interior space or cavity are used in some embodiments. In some embodiments, a nanoshell comprising two or more concentric hollow spheres is used. Such a nanoparticle optionally comprises a core, e.g., made of a dielectric material.

In certain embodiments of the invention, at least 1%, or typically at least 5% of the mass or volume of the particle or number of atoms in the particle is contributed by metal atoms. In certain embodiments of the invention, the amount of metal in the particle, or in a core or coating layer comprising a metal, ranges from approximately 5% to 100% by mass, volume, or number of atoms, or can assume any value or range between 5% and 100%.

Certain metal nanoparticles, referred to as plasmon resonant particles, exhibit the well known phenomenon of plasmon resonance. When a metal nanoparticle (usually made of a noble metal such as gold, silver, copper, platinum, etc.) is subjected to an external electric field, its conduction electrons are displaced from their equilibrium positions with respect to the nuclei, which in turn exert an attractive, restoring force. If the electric field is oscillating (as in the case of electromagnetic radiation such as light), the result is a collective oscillation of the conduction electrons in the nanoparticle, known as plasmon resonance (Kelly et al., 2003, J. Phys. Chem. B., 107:668; Schultz et al., 2000, Proc. Natl. Acad. Sci., USA, 97:996; and Schultz, 2003, Curr. Op. Biotechnol., 14:13). The plasmon resonance phenomenon results in extremely efficient wavelength-dependent scattering and absorption of light by the particles over particular bands of frequencies, often in the visible range. Scattering and absorption give rise to a number of distinctive optical properties that can be detected using various approaches including visually (i.e., by the naked eye or using appropriate microscopic techniques) and/or by obtaining a spectrum, such as a scattering spectrum, extinction (scattering+absorption) spectrum, or absorption spectrum from the particle(s).

Features of the spectrum of a plasmon resonant particle (e.g., peak wavelength) depend on a number of factors, including the particle's material composition, the particle's shape and size, the surrounding medium's refractive index or dielectric properties, and the presence of other particles in the vicinity. Selection of particular particle shapes, sizes, and compositions makes it possible to produce particles with a wide range of distinguishable optically detectable properties.

Single plasmon resonant nanoparticles of sufficient size can be individually detected using a variety of approaches. For example, particles larger than about 30 nm in diameter are readily detectable under an optical microscope operating in dark-field. A spectrum from these particles can be obtained, e.g., using a CCD detector or other optical detection device. Despite their small dimensions relative to the wavelength of light, metal nanoparticles can be detected optically because they scatter light very efficiently at their plasmon resonance frequency. An 80 nm particle, for example, would be millions of times brighter than a fluorescein molecule under the same illumination conditions (Schultz et al., 2000, Proc. Natl. Acad. Sci., USA, 97:996). Individual plasmon resonant particles can be optically detected using a variety of approaches including near-field scanning optical microscopy, differential interference microscopy with video enhancement, total internal reflection microscopy, photo-thermal interference contrast, etc. For measurements on a population of cells, a standard spectrometer, e.g., equipped for detection of UV, visible, and/or infrared light, can be used. In certain embodiments of the invention, nanoparticles are optically detected with the use of surface-enhanced Raman scattering (SERS) (Jackson et al, 2004, Proc. Natl. Acad. Sci., USA, 101:17930). Optical properties of metal nanoparticles and methods for synthesis of metal nanoparticles have been reviewed (Link et al., 2003, Annu. Rev. Phys. Chem., 54:331; and Masala et al., 2004, Annu. Rev. Mater. Res., 34:41).

Magnetic nanoparticles are of use in the invention. “Magnetic particles” refers to magnetically responsive particles that contain one or more metals, oxides, and/or hydroxides thereof. Such particles typically react to magnetic force resulting from a magnetic field. A magnetic field can attract or repel particles towards or away from the source of the magnetic field, respectively, optionally causing acceleration or movement in a desired direction in space. A magnetically detectable nanoparticle is a magnetic particle that can be detected as a consequence of its magnetic properties. In some embodiments, a magnetically detectable nanoparticle can be detected within a living cell as a consequence of its magnetic properties.

Magnetic particles may comprise one or more ferrimagnetic, ferromagnetic, paramagnetic, and/or superparamagnetic materials. Useful particles may be made entirely or in part of one or more materials selected from the group consisting of: iron, cobalt, nickel, niobium, magnetic iron oxides, hydroxides such as maghemite (γ-Fe₂O₃), magnetite (Fe₃O₄), feroxyhyte (FeO [OH]), double oxides or hydroxides of two- or three-valent iron with two- or three-valent other metal ions such as those from the first row of transition metals such as Co(II), Mn(II), Cu(II), Ni(II), Cr(III), Gd(III), Dy(III), Sm(III), mixtures of the afore-mentioned oxides or hydroxides, and mixtures of any of the foregoing. See, e.g., U.S. Pat. No. 5,916,539 for suitable synthesis methods for certain of these particles. Additional materials that may be used in magnetic particles include yttrium, europium, and vanadium.

A magnetic particle may contain a magnetic material and one or more nonmagnetic materials, which may be a metal or a nonmetal. In certain embodiments of the invention, a magnetic particle is a composite particle comprising an inner core or layer containing a first material and an outer layer or shell containing a second material, wherein at least one of the materials is magnetic. Optionally both of the materials are metals. In one embodiment, a magnetic nanoparticle is an iron oxide nanoparticle, e.g., the particle has a core of iron oxide. Optionally the iron oxide is monocrystalline. In one embodiment, the nanoparticle is a superparamagnetic iron oxide nanoparticle, e.g., the particle has a core of superparamagnetic iron oxide.

In certain embodiments of the invention, nanoparticles may comprise a bulk material that is not intrinsically fluorescent, luminescent, plasmon resonant, or magnetic, but may comprise one or more fluorescent, luminescent, or magnetic moieties. For example, a nanoparticle may comprise quantum dots, fluorescent or luminescent organic molecules, or smaller particles of a magnetic material. In some embodiments, an optically detectable moiety such as a fluorescent or luminescent dye, etc., is entrapped, embedded, or encapsulated by a nanoparticle core and/or coating layer. In some embodiments, an optically detectable moiety such as a fluorescent or luminescent dye, etc., is conjugated to a nanoparticle.

Cargo Entities

In some embodiments, inventive TSACs may optionally comprise a cargo entity. Cargo entities can be conjugated to monomeric units using techniques known in the art. In some embodiments, a cargo entity is a diagnostic and/or therapeutic agent to be delivered. In some embodiments, a cargo entity is a substance that does not require TSAC self-assembly to be active and/or effective. In some embodiments, such a cargo entity may be conjugated to a TSAC and made available to a target site only upon self-assembly of the TSACs to which the cargo entity is conjugated.

In some embodiments, a cargo entity is a substance that, by itself, has little to no desired effect. However, upon aggregation (e.g., upon interaction of complementary binding moieties), cargo entities can interact to achieve a desired result (e.g. magnetic, optical, or fluorescent properties, as described herein).

In some embodiments, each monomeric unit of a TSAC comprises one or more cargo entities. In some embodiments, each monomeric unit of a TSAC comprises exactly one cargo entity. In some embodiments, some of the monomeric units of a TSAC comprise one or more cargo entities. In some embodiments, some of the monomeric units of a TSAC do not comprise any cargo entities.

One of ordinary skill in the art will appreciate that any cargo entity can be delivered by the compositions and methods of the present invention. In some embodiments, cargo entities may include any molecule, material, substance, or construct that may be transported into a cell by conjugation to a nano- or micro-structure. Typically, cargo entities will comprise at least two complementary cargo domains, such that each alone has little to no desired effect, but when combined have an increased effect. By “complementary cargo domain” is meant that a first cargo domain complements a second cargo domain to become “activated.” A cargo entity may comprise one or more cargo domains. A cargo domain may be, for example, a fluorescent moiety, such as a fluorescent moiety that can undergo fluorescence resonance energy transfer (FRET) and/or bioluminescence resonance energy transfer (BRET). In some embodiments, FRET and/or BRET occur through assembly of an acceptor fluorophore and a donor fluorophore. Exemplary fluorophores that are suitable for FRET include, but are not limited to, quantum dots, molecular beacons, organic fluorophores, etc. Exemplary fluorophores that are suitable for BRET include, but are not limited to, bioluminescent proteins (e.g., luciferase), quantum dots, molecular beacons, organic fluorophores, etc.

Single- and Multi-Component TSANs

In some embodiments, TSANs are “single-component” systems. In other words, TSACs of a “single component” TSAN comprise monomeric units and/or cargo entities that are all identical to one another. To give but a few examples, monomeric units that are suitable for use in single-component TSANs may include metal nanoparticles (e.g. gold, silver, iron, cobalt, zinc, cadmium, nickel, gadolinium, chromium, copper, manganese, palladium, tin, alloys thereof, and/or oxides thereof).

Any cargo entity can be used in single-component TSANs, such as antigens, ligands, receptors, metal particles, etc. To give but one example, a TSAC of a single-component TSAN may comprise a monomeric unit conjugated to a receptor. Upon TSAC self-assembly, the multi-valent display of receptors could result in activation of a receptor and/or receptor complex on the surface of a cell that only occurs with multi-valency. In particular, recognition of B-cell antigen by B-cells of the immune system depends upon such multi-valency.

Alternatively or additionally, dendrimers are suitable for use in single-component TSANs. Dendrimers are fully synthetic macromolecules comprising branched, repeating units in layers emanating radially from a point-like core. In general, properties of dendrimers are determined by the functional groups on the dendrimer surface. Some dendrimers can act as proton sponges. A critical amount of hydrogen-accepting moieties (e.g. dendrimers and/or other proton sponge polymers) can break down endosomes and facilitate endosomal escape and/or cellular toxicity. Thus, the present invention encompasses the recognition that inventive TSANs may be used to construct proton sponges (e.g. dendrimers) of sufficient hydrogen-accepting capacity to break down endosomes.

In some embodiments, TSANs are “two-component” or “multi-component” systems. In other words, TSACs of a “two-component” or “multi-component” TSAN comprises monomeric units and/or cargo entities that are not all identical to one another. In some embodiments, a TSAN comprises two populations of TSACs, wherein each population comprises a different monomeric unit. In some embodiments, a TSAN comprises more than two populations of TSACs, wherein each population comprises a different monomeric unit. In some embodiments, a TSAN comprises two populations of TSACs, wherein each population comprises a different cargo entity. In some embodiments, a TSAN comprises more than two populations of TSACs, wherein each population comprises a different cargo entity. In some embodiments, a TSAN comprises more than two populations of TSACs, wherein each population comprises a different monomeric unit and a different cargo entity. In some embodiments, a TSAN comprises more than two populations of TSACs, wherein each population comprises a different monomeric unit and a different cargo entity.

For example, a TSAN might comprise two populations of TSACs: a first population which comprises a cargo entity useful for gaining entry into cells, and a second population which comprises a cargo entity useful for performing a cytoplasmic function (e.g. an enzyme). Prior to assembly, neither TSAC population on its own can performed the desired cytoplasmic function: the first TSAC population can gain entry into the cell, but lacks the cytoplasmic function activity; and the second TSAC population is capable of performing the cytoplasmic function, but cannot gain entry into the cell. However, upon self-assembly, the TSAC aggregate can gain entry into the cell and perform the desired cytoplasmic function.

In some embodiments, multi-component TSANs are utilized to facilitate the delivery of pro-drugs to a subject. In such a system, one population of TSACs comprises a pro-drug, and a second population of TSACs comprises an activator. TSAC self-assembly increases the effective concentration of activator seen by the pro-drug and increases the effective concentration of pro-drug seen by the activator, thereby increasing the kinetics of pro-drug activation.

In some embodiments, one population of TSACs comprises a quantum dot, and a second population of TSACs comprises a gold particle. TSAC self-assembly brings the quantum dot and gold particle together, enhancing the overall plasmon resonance and/or fluorescence. In some embodiments, the plasmon resonance and/or fluoresence of the TSAC aggregate exceeds the sum of the plasmon resonance and/or fluorescence of the individual TSACs.

In some embodiments, multi-component TSANs are utilized to perform electrochemistry and/or construct circuits and/or sensors. In some embodiments, such a system comprises combinations of conductive and/or semiconductive components (e.g. quantum dots, carbon nanotubes, gold, silver rods and/or particles, magnetic micro- and/or nano-particles, etc.).

In some embodiments, multi-component TSANs are utilized to trigger assembly of transfection reagents. In some embodiments, assembly of transfection reagents may promote enhanced entry into cells. In some embodiments, assembly can promote enhanced escape from endosomes. In some embodiments, transfection reagents may be assembled with DNA, RNA, intracellular toxins, etc. in order to promote delivery of the DNA, RNA, intracellular toxin, etc. to a target cell.

In some embodiments, multi-component TSANs are used to trigger activation of a nanoparticle. To give but one example, one population of TSACs may comprise a liposome, and a second population of TSACs may comprise a lipase. TSAC self-assembly brings the liposome and lipase together, allowing the lipase to act on the liposome. Such a system may be useful, for example, for releasing cargo encapsulated by the liposome.

In some embodiments, multi-component TSANs are used to deliver a cargo entity to a target site in vivo. To give but one example, one population of TSACs may comprise an entity that facilitates targeting of the TSAC assembly to a cell, and a second population of TSACs may comprise a cargo entity to be delivered to the cell. TSAC self-assembly brings the targeting entity and the cargo entity together, allowing for efficient, targeted delivery of the cargo entity.

### Complementary Binding Moieties

Inventive TSACs generally comprise one or more monomeric units and one or more complementary binding moieties. In general, complementary binding moieties are sets of molecules, substances, moieties, entities, and/or agents that are capable of self-recognition and association. One of ordinary skill in the art will appreciate that any complementary binding moiety can be used in accordance with the present invention. Exemplary complementary binding moieties include, but are not limited to, ligands and anti-ligands (e.g. streptavidin and biotin), ligands and receptors (e.g. small molecule ligands and their receptors), antibodies and antigens, phage display-derived peptides, complementary nucleic acids (e.g. DNA hybrids, RNA hybrids, DNA/RNA hybrids, etc.), and aptamers. In some embodiments, complementary binding moieties include streptavidin and biotin. Other exemplary complementary binding moieties include, but are not limited to, moieties exhibiting complementary charges, hydrophobicity, hydrogen bonding, covalent bonding, Van der Waals forces, reactive chemistries, electrostatic interactions, magnetic interactions, etc.

Complementary binding moieties may be attached to monomeric units such that one set of monomeric units is coated with a ligand (e.g., biotin), and another set of monomeric units is coated with the corresponding anti-ligand (e.g., streptavidin). Alternatively or additionally, complementary binding moieties may be added such that all particles are coated with both.

In some embodiments, complementary binding moieties are not able to interact with one another until they have been activated by a trigger. In some embodiments, the trigger causes one or more of the complementary binding moieties to be modified in such a way to allow for the complementary binding moieties to interact with one another. Exemplary modifications include, but are not limited to, phosphorylation, glycosylation, methylation, acetylation, myristoylization, nucleic acid extension via polymerase, attachment of reduced glutathione, etc. To give but one example, complementary binding moieties A and B are capable of interacting with one another, but only when both A and B are phosphorylated. A TSAC comprises a monomeric unit conjugated to either unphosphorylated A or B. Thus, a trigger that would allow A and B to interact might be a kinase which phosphorylates both A and B.

In some embodiments, one or more complementary binding moieties may be cloaked by a blocking agent, wherein the blocking agent prevents the complementary binding moieties from interacting with one another. In such a system, complementary binding moieties are allowed to interact when blocking agent is removed.

### Blocking Agents

TSACs may optionally comprise a blocking agent which blocks the ability of complementary binding moieties to interact with one another prior to a desired condition or time. In certain embodiments, blocking molecules may mask, block, cloak, and/or sterically inhibit the activity, self-recognition, and/or self-assembly of complementary binding moieties. In specific embodiments, the presence of a blocking agent on the surface of a TSAC sterically inhibits self-assembly until removal of the blocking agent by cleavage of the cleavable substrate. Once blocking agents are removed, TSACs are able to self-assemble. In some embodiments, self-assembly causes accumulation and immobilization of TSAC aggregates at the site of activation and self-assembly. In some embodiments, self-assembly may activate diagnostic and therapeutic agents as described herein.

Methods have been previously described which utilize charge neutralization (e.g. anions on the end of a cationic sequence) as a “blocking agent.” The present invention encompasses the recognition that steric shielding provides more stable particles which avoid reticuloendothelial system (RES) uptake and have longer circulation times in vivo.

The present invention encompasses the recognition that emergent properties which result from self-assembly of monomeric units mediated by enzymatic uncloaking or unshielding of a blocking agent can be used for diagnostic and/or therapeutic purposes.

Alternatively or additionally, blocking agents may serve to prevent non-specific binding of inventive conjugates to proteins in serum, in the extracellular matrix, or on cell membranes. In some embodiments, blocking agents may provide protection from reticulo-endothelial system (RES) uptake before conjugates are cleaved.

Examples of blocking agents include, but are not limited to, polaxamines, poloxamers, polyethylene glycol (PEG), peptides, or other synthetic polymers of sufficient length and density to both mask self-assembly and provide protection against non-specific adsorption, opsonization, and RES uptake. In some embodiments, a blocking agent is a PEG chain. In some embodiments, the PEG chain is approximately 2.5, approximately 5, approximately 7.5, approximately 10, approximately 15, approximately 20, or approximately 25 kDa.

Cleavable Linkers

In some embodiments, a blocking agent is conjugated to a complementary binding moiety or to a monomeric unit by a cleavable linker (e.g., protease cleavable peptide). Cleavable linkers of the invention may be cleaved via any form of cleavable chemistry. Exemplary cleavable linkers include, but are not limited to, protease cleavable peptide linkers, nuclease sensitive nucleic acid linkers, lipase sensitive lipid linkers, glycosidase sensitive carbohydrate linkers, pH sensitive linkers, hypoxia sensitive linkers, photo-cleavable linkers, heat-labile linkers, enzyme cleavable linkers, ultrasound-sensitive linkers, x-ray cleavable linkers, etc.

In certain specific embodiments, a cleavable linker is a protease cleavable peptide linker. In certain specific embodiments, a cleavable linker is a pH sensitive linker. In certain specific embodiments, a cleavable linker is a glycosidase sensitive linker. In certain specific embodiments, a cleavable linker is a nuclease sensitive linker. In certain specific embodiments, a cleavable linker is a lipase sensitive linker. In certain specific embodiments, a cleavable linker is a photo-cleavable linker.

A cleavable linker typically comprises between approximately 2 to approximately 1000 atoms, between approximately 2 to approximately 750 atoms, between approximately 2 to approximately 500 atoms, between approximately 2 to approximately 250 atoms, between approximately 2 to approximately 100 atoms, or between about 6 to about 30 atoms.

In some embodiments, cleavable linkers include amino acid residues and may comprise a peptide linkage of between approximately 1 to approximately 30, between approximately 2 to approximately 20, or between approximately 2 to approximately 10 amino acid residues.

In some embodiments, cleavable linkers include nucleic acid residues and may comprise between approximately 1 to approximately 30, between approximately 2 to approximately 20, or between approximately 2 to approximately 10 nucleic acid residues joined by phosphodiester linkages.

In some embodiments, cleavable linkers include carbohydrates. Carbohydrates may be monosaccharides, disaccharides, and/or polysaccharides. In some embodiments, carbohydrate linkers may comprise between approximately 1 to approximately 30, between approximately 2 to approximately 20, or between approximately 2 to approximately 10 monosaccharides joined by glycosidic linkages.

A cleavable linker suitable for the practice of the invention may be a flexible linker. For example, a cleavable linker suitable for the practice of the invention may be a flexible linker which is approximately 6 to approximately 24 atoms in length. In some embodiments of the invention, a cleavable linker includes an aminocaproic acid (also termed aminohexanoic acid) linkage.

In certain embodiments, a cleavable linker may include a disulfide bridge (Oishi et al., 2005, J. Am. Chem. Soc., 127:1624). In some embodiments, a cleavable linker may include a transition metal complex that falls apart when the metal is reduced. In specific embodiments, a cleavable linker may include an acid-labile thioester.

After cleavage of peptide linkers, blocking agents are removed from TSACs, thereby exposing pairs of complementary binding moieties, allowing interaction. Upon interaction, cargo entities comprising complementary cargo domains (e.g., diagnostic and/or therapeutic) can interact to effectuate any desired result. A cleavable linker is typically cleavable under physiological conditions, allowing transport of cargo into living cells or tissue.

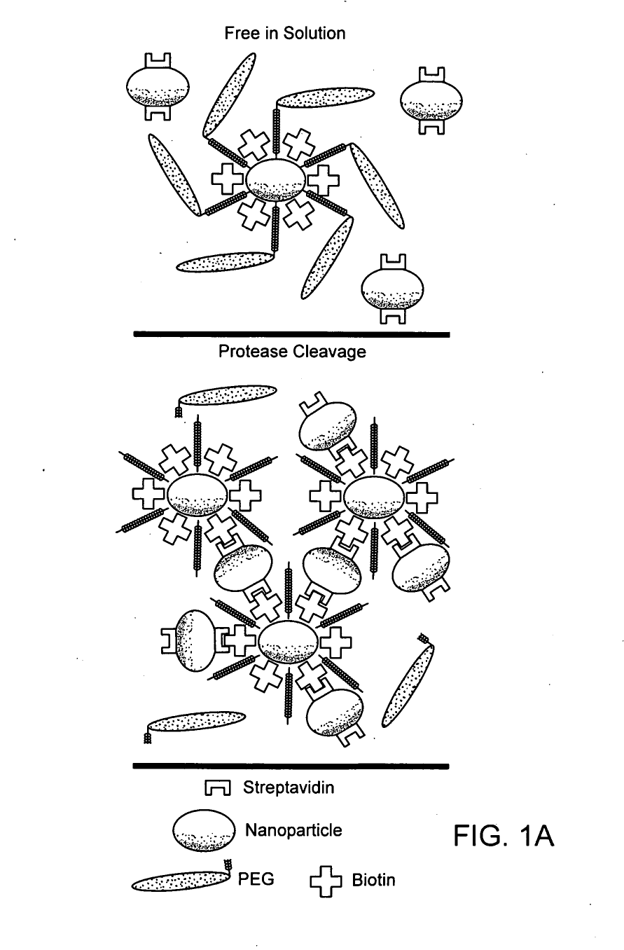

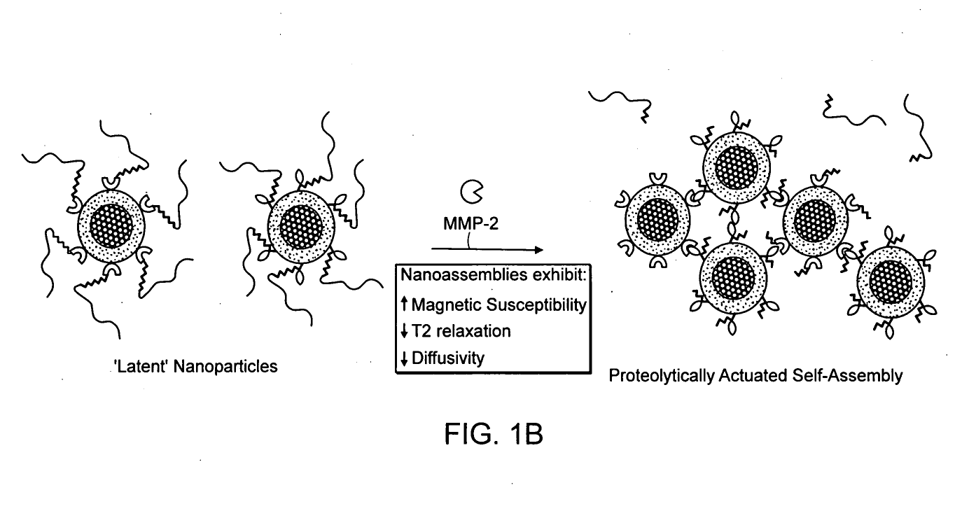

A simple example of a composition and method of the invention is shown in FIG. 1A. A monomeric unit (e.g., a nanoparticle), complementary binding moieties (e.g., streptavidin and biotin), a blocking agent (e.g., PEG), and a protease cleavable linker are shown. As depicted in FIG. 1A, only the biotin coated nanoparticle is modified with the blocking agent, PEG. Upon proteolyic cleavage of the blocking agent (e.g. PEG), the complementary binding moieties (e.g., streptavidin and biotin) interact thereby causing the nanostructures to self-assemble to form a larger aggregate.

Cleavage typically occurs at sites where corresponding triggers are present. For example, when a TSAC comprising a blocking agent is introduced into a region of high enzyme expression (e.g. tumor interstitium where a high concentration of MMPs are present, since MMPs are upregulated in many types of tumors), extracellular cleavage of the linker leads to separation of TSAC and blocking agent. Whereas, without the presence of MMPs, the blocking agent remains attached to the TSAC. As a result, complementary binding moieties of TSACs are allowed to interact with one another when TSACs reach tumor sites in vivo.

In some embodiments, a cleavable linker may be configured to be cleaved under conditions associated with the extracellular space. In certain embodiments of the invention, a cleavable linker may be configured to be cleaved under conditions associated with cell damage, tissue damage, or disease. Such conditions include, for example, acidosis; the presence of intracellular enzymes (that are normally confined within cells), including necrotic conditions (e.g., cleaved by calpains or other proteases that spill out of necrotic cells); hypoxic conditions, such as a reducing environment; thrombosis (e.g., a linker may be cleavable by thrombin or by another enzyme associated with the blood clotting cascade); immune system activation (e.g., a linker may be cleavable by action of an activated complement protein); or other condition associated with disease or injury.

In certain specific embodiments, a cleavable linker may be configured for cleavage by an enzyme, such as a matrix metalloproteinase (MMP). Any MMP can be used in accordance with the present invention (e.g. MMP-2, MMP-7, etc.). In some embodiments of the invention, cleavable linker may include the amino acid sequence PLGLAG or may include the amino acid sequence EDDDDKA.

Exemplary enzymes which may cleave a cleavable linker include, but are not limited to, urokinase plasminogen activator (uPA), lysosomal enzymes, cathepsins (e.g. cathespin S, cathespin K), prostate-specific antigen, herpes simplex virus protease, cytomegalovirus protease, thrombin, caspases (e.g. caspase-1, caspase-2, caspase-3, etc.), and interleukin 1-β converting enzyme, etc. In some embodiments, the cleavable peptide sequence, protease, and disease to be treated and/or diagnosed are selected from Table I (adapted from Funovics et al., 2003, Anal. Bioanal. Chem., 377:956; and Harris et al., 2006, Angew. Chem. Int. Ed., 45:3161):

TABLE 1
Peptide sequences cleavable by proteases

| Target protease | Disease | Substrate Peptide |
|---|---|---|
| Cathepsin B | Cancer | K•K |
| PSA | Prostate cancer | HSSKLQ• |
| Cathepsin D | Breast cancer | PICF•F |
| MMP-2 | Metastases | GPLG•VRG |
| HIV protease | HIV | GVSQNY•PIVG |
| HSV protease | HSV | LVLA•SSSFGY |
| Caspase-3 | Apoptosis | DEVD• |
| Caspase-1 (ICE) | Apoptosis | WEHD• |
| Thrombin | Cardiovascular | F(Pip*)R•S |

*Pip: pipeloic acid
•(dot): indicates cleavage site.

In some embodiments, TSACs are associated with one or more cell-penetrating peptides and subsequently associated with polyethylene glycol (PEG), which can serve to cloak TSACs and cell-penetrating peptides. In some embodiments, PEG is covalently associated with TSACs and/or cell-penetrating peptides. In some embodiments, PEG is covalently conjugated to TSACs and/or cell-penetrating peptides by a peptide linker. In some embodiments, this peptide linker is a recognition signal for cleavage by a protease. In some embodiments, the protease is one that is expressed in tumor cells. In certain embodiments, the protease is one that is expressed at higher levels in tumor cells relative to non-tumor cells. When the TSAC associated with PEG and cell-penetrating peptides reaches a tumor cell, the protease cleaves the peptide at the recognition site, thereby unmasking the cell-penetrating peptide and allowing the TSAC associated with cell-penetrating peptides to enter the cell. In certain embodiments, the TSAC is further associated with an agent to be delivered, and this agent is delivered upon cellular entry.

### Methods of Manufacturing TSACs

Inventive TSACs may be manufactured using any available method. Methods of forming monomeric units (e.g. metallic nanoparticles or microparticles) are known in the art. For example, aqueous and organic solvent syntheses for monodisperse semiconductor, conductive, magnetic, organic, and other nanoparticles have been developed elsewhere (Pellegrino et al., 2005, Small, 1:48; Murray et al., 2000, Ann. Rev. Mat. Sci., 30:545; and Trindade et al., 2001, Chem. Mat., 13:3843). Alternatively or additionally, particulate formulations can be formed by methods as milling, microfabrication, nanofabrication, sacrificial layers, etc., which are known in the art (Haynes et al., 2001, J. Phys. Chem., 105:5599).

In some embodiments, inventive TSACs comprise one or more monomeric units and one or more complementary binding moieties. In certain embodiments, inventive TSACs comprise one or more monomeric units, one or more complementary binding moieties, and one or more blocking agents. In certain specific embodiments, inventive TSACs comprise one or more monomeric units, one or more complementary binding moieties, one or more blocking agents, and one or more cargo entities.

In some embodiments, the monomeric unit and the complementary binding moiety are physically conjugated. In some embodiments, the monomeric unit and the blocking agent are physically conjugated. In some embodiments, the monomeric unit and the cargo entity are physically conjugated. In some embodiments, the complementary binding moiety and the blocking agent are physically conjugated. In some embodiments, the complementary binding moiety and the cargo entity are physically conjugated. In some embodiments, the blocking agent and the cargo entity are physically conjugated. In certain specific embodiments, the monomeric unit, complementary binding moiety, blocking agent, and cargo entity are physically conjugated.

Physical conjugation can be achieved in a variety of different ways. Physical conjugation may be covalent or non-covalent. The monomeric unit, complementary binding moiety, blocking agent and/or cargo entity may be directly conjugated to one another, e.g., by one or more covalent bonds, or may be conjugated by means of one or more linkers. In one embodiment, the linker forms one or more covalent or non-covalent bonds with the monomeric unit and one or more covalent or non-covalent bonds with the complementary binding moiety, thereby attaching them to one another. In some embodiments, a first linker forms a covalent or non-covalent bond with the monomeric unit and a second linker forms a covalent or non-covalent bond with the complementary binding moiety. The two linkers form one or more covalent or non-covalent bond(s) with each other.

In one embodiment, the linker forms one or more covalent or non-covalent bonds with the monomeric unit and one or more covalent or non-covalent bonds with the blocking agent, thereby attaching them to one another. In some embodiments, a first linker forms a covalent or non-covalent bond with the monomeric unit and a second linker forms a covalent or non-covalent bond with the blocking agent. The two linkers form one or more covalent or non-covalent bond(s) with each other.

In one embodiment, the linker forms one or more covalent or non-covalent bonds with the blocking agent and one or more covalent or non-covalent bonds with the complementary binding moiety, thereby attaching them to one another. In some embodiments, a first linker forms a covalent or non-covalent bond with the blocking agent and a second linker forms a covalent or non-covalent bond with the complementary binding moiety. The two linkers form one or more covalent or non-covalent bond(s) with each other.

In one embodiment, the linker forms one or more covalent or non-covalent bonds with the monomeric unit and one or more covalent or non-covalent bonds with the cargo entity, thereby attaching them to one another. In some embodiments, a first linker forms a covalent or non-covalent bond with the monomeric unit and a second linker forms a covalent or non-covalent bond with the cargo entity. The two linkers form one or more covalent or non-covalent bond(s) with each other.

In some embodiments, the linker is a cleavable linker. To give but a few examples, cleavable linkers include protease cleavable peptide linkers, nuclease sensitive nucleic acid linkers, lipase sensitive lipid linkers, glycosidase sensitive carbohydrate linkers, pH sensitive linkers, hypoxia sensitive linkers, photo-cleavable linkers, heat-labile linkers, enzyme cleavable linkers, ultrasound-sensitive linkers, x-ray cleavable linkers, etc. In some embodiments, the linker is not a cleavable linker.

Any of a variety of methods can be used to conjugate a linker (e.g. a biomolecule such as a polypeptide, carbohydrate, or nucleic acid) to a nanoparticle (e.g. TSAC). General strategies include passive adsorption (e.g., via electrostatic interactions), multivalent chelation, high affinity non-covalent binding between members of a specific binding pair, covalent bond formation, etc. (Gao et al. Curr. Op. Biotechnol., 16:63).

A bifunctional cross-linking reagent can be employed. Such reagents contain two reactive groups, thereby providing a means of covalently conjugating two target groups. The reactive groups in a chemical cross-linking reagent typically belong to various classes of functional groups such as succinimidyl esters, maleimides, and pyridyldisulfides. Exemplary cross-linking agents include, e.g., carbodiimides, N-hydroxysuccinimidyl-4-azidosalicylic acid (NHS-ASA), dimethyl pimelimidate dihydrochloride (DMP), dimethylsuberimidate (DMS), 3,3′-dithiobispropionimidate (DTBP), N-Succinimidyl 3-[2-pyridyldithio]-propionamido (SPDP), succimidyl α-methylbutanoate, biotinamidohexanoyl-6-amino-hexanoic acid N-hydroxy-succinimide ester (SMCC), succinimidyl-[(N-maleimidopropionamido)-dodecaethyleneglycol]ester (NH S-PEO12), etc. For example, carbodiimide-mediated amide formation and active ester maleimide-mediated amine and sulfhydryl coupling are widely used approaches.

Common schemes for forming a conjugate involve the coupling of an amine group on one molecule to a thiol group on a second molecule, sometimes by a two- or three-step reaction sequence. A thiol-containing molecule may be reacted with an amine-containing molecule using a heterobifunctional cross-linking reagent, e.g., a reagent containing both a succinimidyl ester and either a maleimide, a pyridyldisulfide, or an iodoacetamide. Amine-carboxylic acid and thiol-carboxylic acid cross-linking, maleimide-sulfhydryl coupling chemistries (e.g., the maleimidobenzoyl-N-hydroxysuccinimide ester (MBS) method), etc., may be used. Polypeptides can conveniently be attached to nanoparticles via amine or thiol groups in lysine or cysteine side chains respectively, or by an N-terminal amino group. Nucleic acids such as RNAs can be synthesized with a terminal amino group. A variety of coupling reagents (e.g., succinimidyl 3-(2-pyridyldithio)propionate (SPDP) and sulfosuccinimidyl-4-(N-maleimidomethyl)cyclohexane-1-carboxylate (sulfo-SMCC) may be used to conjugate the various components of TSACs. Monomeric units can be prepared with functional groups, e.g., amine or carboxyl groups, available at the surface to facilitate conjugation to a biomolecule.

Non-covalent specific binding interactions can be employed. For example, either a nanoparticle or a biomolecule can be functionalized with biotin with the other being functionalized with streptavidin. These two moieties specifically bind to each other non-covalently and with a high affinity, thereby conjugating the nanoparticle and the biomolecule. Other specific binding pairs could be similarly used. Alternately, histidine-tagged biomolecules can be conjugated to nanoparticles conjugated with nickel-nitrolotriaceteic acid (Ni-NTA).

Any biomolecule to be attached to a monomeric unit, complementary binding moiety, blocking agent, and/or cargo entity may include a spacer. The spacer can be, for example, a short peptide chain, e.g., between 1 and 10 amino acids in length, e.g., 1, 2, 3, 4, or 5 amino acids in length, a nucleic acid, an alkyl chain, etc.

For additional general information on conjugation methods and cross-linkers, see the journal Bioconjugate Chemistry, published by the American Chemical Society, Columbus Ohio, PO Box 3337, Columbus, Ohio, 43210; “Cross-Linking,” Pierce Chemical Technical Library, available at the Pierce web site and originally published in the 1994-95 Pierce Catalog, and references cited therein; Wong SS, Chemistry of Protein Conjugation and Cross-linking, CRC Press Publishers, Boca Raton, 1991; and Hermanson, G. T., Bioconjugate Techniques, Academic Press, Inc., San Diego, 1996.

It is to be understood that the compositions of the invention can be made in any suitable manner, and the invention is in no way limited to compositions that can be produced using the methods described herein. Selection of an appropriate method may require attention to the properties of the particular moieties being conjugated.

If desired, various methods may be used to separate TSACs with an attached complementary binding moiety, blocking agent, or cargo domain from TSACs to which the complementary binding moiety, blocking agent, or cargo domain has not become attached, or to separate TSACs having different numbers of complementary binding moieties, blocking agents, or cargo domains attached thereto. For example, size exclusion chromatography, agarose gel electrophoresis, or filtration can be used to separate populations of TSACs having different numbers of moieties attached thereto and/or to separate TSACs from other entities. Some methods include size-exclusion or anion-exchange chromatography.

Any method may be used to determine whether TSAC aggregates have formed, including measuring extinction coefficients, atomic force microscopy (AFM), etc. An extinction coefficient, generally speaking, is a measure of a substance's turbidity and/or opacity. If EM radiation can pass through a substance very easily, the substance has a low extinction coefficient. Conversely, if EM radiation hardly penetrates a substance, but rather quickly becomes “extinct” within it, the extinction coefficient is high. For example, to determine whether TSAC aggregates have formed, EM radiation is directed toward and allowed to pass through a sample. If the sample contains primarily TSAC aggregates, EM radiation will deflect and scatter in a pattern that is different from the pattern produced by a sample containing primarily individual TSACs.

In general, AFM utilizes a high-resolution type of scanning probe microscope and attains resolution of fractions of an Angstrom. The microscope has a microscale cantilever with a sharp tip (probe) at its end that is used to scan a specimen surface. The cantilever is frequently silicon or silicon nitride with a tip radius of curvature on the order of nanometers. When the tip is brought into proximity of a sample surface, forces between the tip and the sample lead to a deflection of the cantilever according to Hooke's law. Typically, a feedback mechanism is employed to adjust the tip-to-sample distance to maintain a constant force between the tip and the sample. Samples are usually spread in a thin layer across a surface (e.g. mica), which is mounted on a piezoelectric tube that can move the sample in the z direction for maintaining a constant force, and the x and y directions for scanning the sample.

In general, forces that are measured in AFM may include mechanical contact force, Van der Waals forces, capillary forces, chemical bonding, electrostatic forces, magnetic forces, Casimir forces, solvation forces, etc. Typically, deflection is measured using a laser spot reflected from the top of the cantilever into an array of photodiodes. Alternatively or additionally, deflection can be measured using optical interferometry, capacitive sensing, or piezoresistive AFM probes.

### Diagnostic and Therapeutic Applications

In some embodiments, a therapeutic amount of an inventive composition is administered to a subject for therapeutic and/or diagnostic purposes. In some embodiments, the amount of TSAN and/or TSAC is sufficient to treat and/or diagnose a disease, condition, and/or disorder. In some embodiments, the invention encompasses “therapeutic cocktails,” including, but not limited to, approaches in which multiple TSANs and/or TSACs are administered.

The invention provides methods and compositions by which TSACs may not only target specific sites in the body of a subject (e.g. specific organs, tissues, cells, etc.), but also be triggered to self-assemble at these sites to activate or amplify the effect of cargo entities such as diagnostic agents (e.g., imaging agents) and/or therapeutics.

TSACs are designed with specific and tunable self-assembling properties and are modified to avoid interacting with themselves, their complement, and non-specific biological materials until they are triggered by an external stimuli. This method provides methods of avoiding non-specific interactions of TSACs with proteins of the serum, extracellular matrix, or cell membranes. This method provides methods of avoiding uptake by the reticulo-endothelial system (RES) before activation at the target site.

Versatility in the mechanism for triggering self-assembly makes this method applicable over a broad range of diagnostic and/or therapeutic applications. For instance, activation by proteases enables targeting to sites of protease upregulation in cancer, thrombosis, atherosclerosis, arthritis, wound healing and the like. Similarly, diseased tissue having low pH and/or hypoxic tissue could be used to trigger self-assembly. Alternatively, self-assembly may be triggered by any form of radiation (e.g., heat, radiofrequency (RF), light, ultrasound, x-ray, etc.)

When administered intravenously, inventive TSACs circulate through blood vessels and may enter lymphatics and extracellular fluids. In areas of high protease expression, such as a tumor, TSACs become activated (e.g., the blocking agent is removed) allowing for interaction of complementary binding partners and assembly of diagnostic and/or therapeutic agents of the invention. Immobilization of self-assembling TSACs may be achieved by size dependant reduction of diffusion of TSAC aggregates through capillaries, lymphatic vessels, and extracellular space after self-assembly occurs. Alternatively or additionally, immobilization may be achieved by TSAC aggregate attachment to existing or pre-targeted complementary binding moieties present at the site of activation.

Self-assembly of TSACs may activate a diagnostic and/or therapeutic agent not available in non-assembled TSACs. In some embodiments, TSAC aggregates are amenable to detection based on unique optical, ultrasonic, MRI relaxivity, or X-ray contrast properties of TSAC aggregates as compared to individual, non-assembled TSACs. Self-assembly activated diagnostics include, but are not limited to, T2 contrast from the association of iron oxide nanoparticles; x-ray, optical, or ultrasound contrast from the periodic structure of an assembled TSAC aggregate; multi-modal imaging from the association of multiple imaging or contrast agents in a single aggregate, etc.

In some embodiments, self-assembly of TSACs results in delivery of a diagnostic and/or therapeutic agent to a cell. Any diagnostic and/or therapeutic agent may be delivered to a cell using the TSACs and/or TSANs described herein. Exemplary agents to be delivered to cells include, but are not limited to, radioactive moieties, radiopaque moieties, paramagnetic moieties, nanoparticles, vesicles, markers, marker enzymes (e.g., horseradish peroxidase, β-galactosidase, and/or any other enzyme suitable for marking a cell), contrast agents (e.g., for diagnostic imaging), chemotherapeutic agents, radiation-sensitizers (e.g., for radiation therapy), peptides and/or proteins that affect the cell cycle, protein toxins, and/or any other cargo suitable for transport into a cell. In some embodiments, inventive methods are used to diagnose cancer. In some embodiments, inventive methods are used to detect the presence and/or location of a tumor.

In one aspect of the invention, a method for the treatment of disease is provided. In some embodiments, the treatment of a disease comprises administering a therapeutically effective amount of inventive TSANs and/or TSACs to a subject in need thereof, in such amounts and for such time as is necessary to achieve the desired result. In certain embodiments of the present invention a “therapeutically effective amount” of an inventive TSAN or TSAC is that amount effective for treating, alleviating, ameliorating, relieving, delaying onset of, inhibiting progression of, reducing severity of, and/or reducing incidence of one or more symptoms or features of a disease, disorder, and/or condition.

Any disease, disorder, and/or condition may be treated using inventive TSANs and/or TSACs. In particular, any disease, disorder, and/or condition that has an inflammatory component may be treated using inventive compositions and methods. Exemplary diseases, disorders, and/or conditions that may be treated include, but are not limited to, cancer, atherosclerosis, arthritis, wounds, renal disease, chronic obstructive pulmonary disease, autoimmune disorders (e.g. diabetes, lupus, multiple sclerosis, psoriasis, rheumatoid arthritis, etc.), clotting disorders, angiogenic disorders (e.g., macular degeneration), viral/bacterial infections, sepsis, thrombosis, etc. In some embodiments, inventive TSANs and/or TSACs are used to treat a cell proliferative disorder. In some embodiments, for example, a therapeutically effective amount of an inventive TSAN and/or TSAC is that amount effective for inhibiting survival, growth, and/or spread of a tumor.

The present invention provides improved methods of delivery of therapeutic agents. For example, the present invention provides protease or pH mediated delivery for more potent therapeutics and/or diagnostics. In some embodiments, the present invention provides radiation-directed assembly and immobilization of therapeutics and/or diagnostics.

In certain embodiments, the present invention provides triggered assembly to perform combinatorial chemistries such as bringing pro-drug and activator into close proximity by the assembly of two different cargo domain carrying TSACs.

In some embodiments, the invention provides delivery of increased therapeutic dosages to single points, increased specificity of drug release and activity, and/or external monitoring of drug accumulation.

To give but one specific example, one TSAC, carrying a cargo entity (e.g. an activator) and another TSAC, carrying a different cargo entity (e.g. a prodrug), each having complementary binding moieties, become closely associated upon triggered assembly, causing the activation of the prodrug at the site of self-assembly. In some embodiments, such a system, by providing for localized activation of a prodrug, can be used to permit the delivery of a drug that is toxic in its active form.

Self-assembly activated therapeutics include, but are not limited to, activation of a drug from association of prodrug- and activator-carrying TSACs; activation of photo-dynamic therapy (PDT) from association of PDT- and bioluminescent-carrying TSACs; creation of single magnetic moment aggregates from the assembly of super-paramagnetic moment TSACs for subsequent targeting of super-paramagnetic TSACs to a diseased site, etc.

In one aspect of the invention, a method for the diagnosis of disease (e.g. cancer) is provided. In some embodiments, the diagnosis of a disease comprises administering a therapeutically effective amount of inventive TSANs and/or TSACs to a subject in need thereof, in such amounts and for such time as is necessary to achieve the desired result. In certain embodiments of the present invention a “therapeutically effective amount” of an inventive TSAN or TSAC is that amount effective for detecting and/or measuring the presence of one or more symptoms or features of a disease (e.g. cancer). In some embodiments, for example, a therapeutically effective amount of an inventive TSAN or TSAC is that amount effective for detecting the presence and/or determining the location of a tumor.

Following administration to a subject, TSACs can be detected under conditions that allow for detection of an aggregate of TSACs, but do not allow for detection of TSACs that have not undergone self-assembly. Such detection can provide an indication of the presence and/or distribution of a trigger which activates self-assembly. To give but one example, administration of a TSAC which is capable of self-assembly upon activation by a MMP can be useful in the detection of tumors. MMPs are often upregulated in tumors, thus, detection of TSAC aggregates indicates that individual TSACs have come into contact with MMPs, potentially near the location of a tumor.

The present invention provides improved diagnostic methods. For example, the invention provides improved methods of molecular imaging. In some embodiments, the invention provides the amplification of the resolution of conventional targeted imaging. Alternatively or additionally, the invention provides aggregation-specific imaging of protease activity in vivo or in whole blood samples.

Detection can take place at any suitable time following administration. In one embodiment, a tissue sample (e.g., a tissue section) is obtained from a subject and examined by any of the techniques described herein. Alternatively or additionally, individual cells can be isolated from a subject and examined, sorted, or further processed. In vivo imaging techniques such as fluorescence imaging can be employed to detect nanoparticles in a living subject (Gao et al., 2004, Nat. Biotechnol., 22:969). In vivo administration provides the potential for rapidly evaluating the ability of different delivery vehicles to enhance uptake of an agent in a living organism. In addition to detecting aggregates of TSACs, conventional immunostaining or other techniques can be employed, e.g., to gather information about the effect of the TSAC aggregate on the subject, etc.

The present invention provides in vitro applications for inventive TSACs and/or TSANs. In vitro use contemplates targeting of a substance in cell-culture assays, chemical or biowarfare detection, drug discovery, enzyme activity, etc. In some embodiments, inventive TSACs and/or TSANs can be used for patterned self assembly on a surface to build bottom-up nanostructures (e.g., light or heat triggered self-assembly on a surface or over cells).

In some embodiments, self-assembly of inventive TSACs is an irreversible process. In some embodiments, self-assembly of inventive TSACs is a reversible process. The present invention encompasses the recognition that the inventive conjugates may be used to reversibly sense multiple triggers (e.g. enzyme activities). In some embodiments, TSACs can alternate between separate and self-assembled states. In some embodiments, such alternation is indicative of the environment surrounding the TSACs. In particular, such alternation is indicative of the presence or absence of one or more triggers and/or is indicative of the relative amounts of one or more triggers. For example, an inventive TSAN comprises TSACs that can self-assemble in the presence of kinase activity and re-disperse in the presence of phosphatase activity. Such a system can provide a method for monitoring kinase and phosphatase activities by self-assembling as TSACs become phosphorylated and disassembling as phosphates are removed.

The present invention encompasses the recognition that by conjugating blocking agents to each TSAC via unique cleavable linkers (e.g. unique protease substrates), assembly can be restricted to occur only in the presence of both triggers (e.g. two proteases which recognize the unique protease substrates). Thus, inventive methods can be used to simultaneously detect the presence and/or location of two or more triggers.

The present invention encompasses the recognition that by conjugating blocking agents to one population of TSACs with tandem unique cleavable linkers (e.g. two or more unique protease substrates in tandem), assembly can be restricted to occur in the presence of either or both triggers (e.g. one or more proteases which recognize one or more of the unique protease substrates).

### Administration

The compositions, according to the method of the present invention, may be administered using any amount and any route of administration effective for treatment. The exact amount required will vary from subject to subject, depending on the species, age, and general condition of the subject, the severity of the infection, the particular composition, its mode of administration, its mode of activity, and the like. The compositions of the invention are typically formulated in dosage unit form for ease of administration and uniformity of dosage. It will be understood, however, that the total daily usage of the compositions of the present invention will be decided by the attending physician within the scope of sound medical judgment. The specific therapeutically effective dose level for any particular subject or organism will depend upon a variety of factors including the disorder being treated and the severity of the disorder; the activity of the specific compound employed; the specific composition employed; the age, body weight, general health, sex and diet of the subject; the time of administration, route of administration, and rate of excretion of the specific compound employed; the duration of the treatment; drugs used in combination or coincidental with the specific compound employed; and like factors well known in the medical arts.

The pharmaceutical compositions of the present invention may be administered by any route. In some embodiments, the pharmaceutical compositions of the present invention are administered variety of routes, including oral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, enteral, sublingual; by intratracheal instillation, bronchial instillation, and/or inhalation; and/or as an oral spray, nasal spray, and/or aerosol. Specifically contemplated routes are systemic intravenous injection, regional administration via blood and/or lymph supply, and/or direct administration to an affected site. In general the most appropriate route of administration will depend upon a variety of factors including the nature of the agent (e.g., its stability in the environment of the gastrointestinal tract), the condition of the subject (e.g., whether the subject is able to tolerate oral administration), etc. At present the oral and/or nasal spray and/or aerosol route is most commonly used to deliver therapeutic agents directly to the lungs and/or respiratory system. However, the invention encompasses the delivery of the inventive pharmaceutical composition by any appropriate route taking into consideration likely advances in the sciences of drug delivery.

In certain embodiments, the compounds of the invention may be administered orally or parenterally at dosage levels sufficient to deliver from about 0.001 mg/kg to about 100 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic effect. The desired dosage may be delivered three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations).

It will be appreciated that the TSANs, TSACs, and pharmaceutical compositions of the present invention can be employed in combination therapies. The particular combination of therapies (therapeutics or procedures) to employ in a combination regimen will take into account compatibility of the desired therapeutics and/or procedures and the desired therapeutic effect to be achieved. It will be appreciated that the therapies employed may achieve a desired effect for the same purpose (for example, an inventive TSAN and/or TSAC useful for detecting tumors may be administered concurrently with another agent useful for detecting tumors), or they may achieve different effects (e.g., control of any adverse effects).

Pharmaceutical compositions of the present invention may be administered either alone or in combination with one or more other therapeutic agents. By “in combination with,” it is not intended to imply that the agents must be administered at the same time and/or formulated for delivery together, although these methods of delivery are within the scope of the invention. The compositions can be administered concurrently with, prior to, or subsequent to, one or more other desired therapeutics or medical procedures. In general, each agent will be administered at a dose and/or on a time schedule determined for that agent. Additionally, the invention encompasses the delivery of the inventive pharmaceutical compositions in combination with agents that may improve their bioavailability, reduce and/or modify their metabolism, inhibit their excretion, and/or modify their distribution within the body.

The particular combination of therapies (therapeutics and/or procedures) to employ in a combination regimen will take into account compatibility of the desired therapeutics and/or procedures and/or the desired therapeutic effect to be achieved. It will be appreciated that the therapies employed may achieve a desired effect for the same disorder (for example, an inventive compound may be administered concurrently with another agent used to treat the same disorder), and/or they may achieve different effects (e.g., control of any adverse effects).

In will further be appreciated that therapeutically active agents utilized in combination may be administered together in a single composition or administered separately in different compositions.

In general, it is expected that agents utilized in combination with be utilized at levels that do not exceed the levels at which they are utilized individually. In some embodiments, the levels utilized in combination will be lower than those utilized individually.

In some embodiments, TSANs and TSACs which are used as diagnostic agents may be used in combination with one or more other diagnostic agents. To give but one example, TSANs and TSACs used to detect tumors may be administered in combination with other agents useful in the detection of tumors. For example, inventive TSANs and TSACs may be administered in combination with traditional tissue biopsy followed by immunohistochemical staining and serological tests (e.g. prostate serum antigen test). Alternatively or additionally, inventive TSANs and TSACs may be administered in combination with a contrasting agent for use in computed tomography (CT) scans and/or MRI.

In some embodiments, TSANs and TSACs which are used as therapeutic agents may be used in combination with other diagnostic. To give but one example, TSANs and TSACs used to treat tumors may be administered in combination with other agents useful in the treatment of tumors. For example, inventive TSANs and TSACs may be administered in combination with traditional chemotherapy, radiation treatment, surgical removal of a tumor, administration of biologics (e.g. therapeutic antibodies), etc.

### Kits

The invention provides a variety of kits for conveniently and/or effectively carrying out methods of the present invention. Inventive kits typically comprise one or more TSANs and/or TSACs. In some embodiments, kits comprise a collection of different TSANs and/or TSACs to be used for different purposes (e.g. diagnostics and/or treatment). Typically kits will comprise sufficient amounts of TSANs and/or TSACs to allow a user to perform multiple treatments of a subject(s) and/or to perform multiple experiments.

Inventive kits may include additional components or reagents. For example, kits may comprise one or more substances (e.g., a small molecule, protein, etc.) that trigger and/or inhibit self-assembly. Kits may comprise one or more control TSANs and/or TSACs, e.g., positive and negative control TSANs and/or TSACs. Other components of inventive kits may include cells, cell culture media, tissue, and/or tissue culture media.

In some embodiments, kits are supplied with or include one or more TSANs and/or TSACs that have been specified by the purchaser.

Inventive kits may comprise instructions for use. For example, instructions may inform the user of the proper procedure by which to prepare a pharmaceutical composition comprising TSANs and/or TSACs and/or the proper procedure for administering the pharmaceutical composition to a subject.

In some embodiments, kits include a number of unit dosages of a pharmaceutical composition comprising TSANs and/or TSACs. A memory aid may be provided, for example in the form of numbers, letters, and/or other markings and/or with a calendar insert, designating the days/times in the treatment schedule in which dosages can be administered. Placebo dosages, and/or calcium dietary supplements, either in a form similar to or distinct from the dosages of the pharmaceutical compositions, may be included to provide a kit in which a dosage is taken every day.

Kits may comprise one or more vessels or containers so that certain of the individual components or reagents may be separately housed. Inventive kits may comprise a means for enclosing the individual containers in relatively close confinement for commercial sale, e.g., a plastic box, in which instructions, packaging materials such as styrofoam, etc., may be enclosed.

In certain embodiments, inventive kits are adaptable to high-throughput and/or automated operation. For example, kits may be suitable for performing assays in multiwell plates and may utilize automated fluid handling and/or robotic systems, plate readers, etc.

Optionally associated with inventive kits may be a notice in the form prescribed by a governmental agency regulating the manufacture, use and/or sale of pharmaceutical products, which notice reflects approval by the agency of manufacture, use and/or sale for human administration.

In some embodiments, inventive kits comprise one or more TSANs and/or TSACs of the invention. In some embodiments, such a kit is used in the diagnosis and/or treatment of a subject suffering from and/or susceptible to a disease, condition, and/or disorder (e.g. cancer). In some embodiments, such a kit comprises (i) a TSAN and/or TSAC that is useful in the treatment of cancer; (ii) a syringe, swab, applicator, etc. for administration of the TSAN and/or TSAC to a subject; and (iii) instructions for use.

The invention provides kits for identifying TSANs and/or TSACs which are useful in treating and/or diagnosing a disease, disorder, and/or condition. In some embodiments, such a kit comprises (i) a TSAN and/or TSAC known to be useful in the diagnosis and/or treatment of a subject suffering from and/or susceptible to a disease, condition, and/or disorder (positive control); (ii) a TSAN and/or TSAC that is known not to be useful in the diagnosis and/or treatment of a subject suffering from and/or susceptible to a disease, condition, and/or disorder (negative control); (iii) a substance (e.g. a small molecule, protein, etc.) that triggers self-assembly (positive control); (iv) a substance (e.g., a small molecule, protein, etc.) that inhibits self-assembly (negative control); (v) cells and/or subjects suffering from and/or susceptible to a disease, disorder, and/or condition of interest and displaying symptoms characteristic of the disease, disorder, and/or condition; (vi) cells and/or subjects not suffering from and/or susceptible to a disease, disorder, and/or condition of interest and not displaying symptoms characteristic of the disease, disorder, and/or condition; (vii) materials to assay the effect of an TSAN and/or TSAC on the symptoms of the disease, disorder, and/or condition displayed by cells and/or subjects; and (viii) instructions for use.

### Pharmaceutical Compositions

The present invention provides inventive triggered self-assembly nanosystems (TSANs) and triggered self-assembly conjugates (TSACs). In some embodiments, the present invention provides for pharmaceutical compositions comprising TSANs and/or TSACs as described herein. Such pharmaceutical compositions may optionally comprise one or more additional therapeutically-active substances. In accordance with one embodiment, a method of administering a pharmaceutical composition comprising inventive antimicrobials to a subject in need thereof is provided. In some embodiments, the compositions are administered to humans. For the purposes of the present invention, the phrase “active ingredient” generally refers to an inventive TSAN and/or TSAC.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions of the invention is contemplated include, but are not limited to, humans and/or other primates; mammals, including commercially relevant mammals such as cattle, pigs, horses, sheep, cats, and/or dogs; and/or birds, including commercially relevant birds such as chickens, ducks, geese, and/or turkeys.

Formulations of the pharmaceutical compositions described herein may be prepared by any method known or hereafter developed in the art of pharmacology. In general, such preparatory methods include the step of bringing the active ingredient into association with a carrier and/or one or more other accessory ingredients, and then, if necessary and/or desirable, shaping and/or packaging the product into a desired single- or multi-dose unit.

A pharmaceutical composition of the invention may be prepared, packaged, and/or sold in bulk, as a single unit dose, and/or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable carrier, and/or any additional ingredients in a pharmaceutical composition of the invention will vary, depending upon the identity, size, and/or condition of the subject treated and further depending upon the route by which the composition is to be administered. By way of example, the composition may comprise between 0.1% and 100% (w/w) active ingredient.

Pharmaceutical formulations of the present invention may additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21^{st} Edition, A. R. Gennaro, (Lippincott, Williams & Wilkins, Baltimore, Md., 2006) discloses various carriers used in formulating pharmaceutical compositions and known techniques for the preparation thereof. Except insofar as any conventional carrier medium is incompatible with a substance or its derivatives, such as by producing any undesirable biological effect or otherwise interacting in a deleterious manner with any other component(s) of the pharmaceutical composition, its use is contemplated to be within the scope of this invention.

In some embodiments, the pharmaceutically acceptable excipient is at least 95%, 96%, 97%, 98%, 99%, or 100% pure. In some embodiments, the excipient is approved for use in humans and for veterinary use. In some embodiments, the excipient is approved by United States Food and Drug Administration. In some embodiments, the excipient is pharmaceutical grade. In some embodiments, the excipient meets the standards of the United States Pharmacopoeia (USP), the European Pharmacopoeia (EP), the British Pharmacopoeia, and/or the International Pharmacopoeia.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in the inventive formulations. Excipients such as cocoa butter and suppository waxes, coloring agents, coating agents, sweetening, flavoring, and perfuming agents can be present in the composition, according to the judgment of the formulator.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and combinations thereof

Exemplary granulating and/or dispersing agents include, but are not limited to, potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and combinations thereof.

Exemplary surface active agents and/or emulsifiers include, but are not limited to, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof.

Exemplary binding agents include, but are not limited to, starch (e.g. cornstarch and starch paste); gelatin; sugars (e.g. sucrose, glucose, dextrose, dextrin, molasses, lactose, lactitol, mannitol); natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan); alginates; polyethylene oxide; polyethylene glycol; inorganic calcium salts; silicic acid; polymethacrylates; waxes; water; alcohol; etc.; and combinations thereof.

Exemplary preservatives may include, but are not limited to, antioxidants, chelating agents, antimicrobial preservatives, antifungal preservatives, alcohol preservatives, acidic preservatives, and other preservatives. Exemplary antioxidants include, but are not limited to, alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite. Exemplary chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and trisodium edetate. Exemplary antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal. Exemplary antifingal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and sorbic acid. Exemplary alcohol preservatives include, but are not limited to, ethanol, polyethylene glycol, phenol, phenolic compounds, bisphenol, chlorobutanol, hydroxybenzoate, and phenylethyl alcohol. Exemplary acidic preservatives include, but are not limited to, vitamin A, vitamin C, vitamin E, beta-carotene, citric acid, acetic acid, dehydroacetic acid, ascorbic acid, sorbic acid, and phytic acid. Other preservatives include, but are not limited to, tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl. In certain embodiments, the preservative is an anti-oxidant. In other embodiments, the preservative is a chelating agent.

Exemplary buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and combinations thereof.

Exemplary lubricating agents include, but are not limited to, magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary oils include, but are not limited to, almond, apricot kernel, avocado, babassu, bergamot, black current seed, borage, cade, chamomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and combinations thereof.

Liquid dosage forms for oral and parenteral administration include, but are not limited to, pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, inventive compositions are mixed with solubilizing agents such an Cremophor, alcohols, oils, modified oils, glycols, polysorbates, cyclodextrins, polymers, and combinations thereof.

Injectable preparations, for example, sterile injectable aqueous or oleaginous suspensions may be formulated according to the known art using suitable dispersing or wetting agents and suspending agents. The sterile injectable preparation may be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of an active ingredient, it is often desirable to slow the absorption of the active ingredient from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are prepared by entrapping the drug in liposomes or microemulsions which are compatible with body tissues.

Compositions for rectal or vaginal administration are typically suppositories which can be prepared by mixing inventive compositions with suitable non-irritating excipients or carriers such as cocoa butter, polyethylene glycol or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or fillers or extenders (e.g. starches, lactose, sucrose, glucose, mannitol, and silicic acid), binders (e.g. carboxymethylcellulose, alginates, gelatin, polyvinylpyrrolidinone, sucrose, and acacia), humectants (e.g. glycerol), disintegrating agents (e.g. agar, calcium carbonate, potato or tapioca starch, alginic acid, certain silicates, and sodium carbonate), solution retarding agents (e.g. paraffin), absorption accelerators (e.g. quaternary ammonium compounds), wetting agents (e.g. cetyl alcohol and glycerol monostearate), absorbents (e.g. kaolin and bentonite clay), and lubricants (e.g. talc, calcium stearate, magnesium stearate, solid polyethylene glycols, sodium lauryl sulfate), and mixtures thereof. In the case of capsules, tablets and pills, the dosage form may comprise buffering agents.

Solid compositions of a similar type may be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes. Solid compositions of a similar type may be employed as fillers in soft and hard-filled gelatin capsules using such excipients as lactose or milk sugar as well as high molecular weight polyethylene glycols and the like.

Dosage forms for topical and/or transdermal administration of a compound of this invention may include ointments, pastes, creams, lotions, gels, powders, solutions, sprays, inhalants and/or patches. Generally, the active ingredient is admixed under sterile conditions with a pharmaceutically acceptable carrier and/or any needed preservatives and/or buffers as may be required. Additionally, the present invention contemplates the use of transdermal patches, which often have the added advantage of providing controlled delivery of a compound to the body. Such dosage forms may be prepared, for example, by dissolving and/or dispensing the compound in the proper medium. Alternatively or additionally, the rate may be controlled by either providing a rate controlling membrane and/or by dispersing the compound in a polymer matrix and/or gel.

Suitable devices for use in delivering intradermal pharmaceutical compositions described herein include short needle devices such as those described in U.S. Pat. Nos. 4,886,499; 5,190,521; 5,328,483; 5,527,288; 4,270,537; 5,015,235; 5,141,496; and 5,417,662. Intradermal compositions may be administered by devices which limit the effective penetration length of a needle into the skin, such as those described in PCT publication WO 99/34850 and functional equivalents thereof. Jet injection devices which deliver liquid vaccines to the dermis via a liquid jet injector and/or via a needle which pierces the stratum corneum and produces a jet which reaches the dermis are suitable. Jet injection devices are described, for example, in U.S. Pat. Nos. 5,480,381; 5,599,302; 5,334,144; 5,993,412; 5,649,912; 5,569,189; 5,704,911; 5,383,851; 5,893,397; 5,466,220; 5,339,163; 5,312,335; 5,503,627; 5,064,413; 5,520,639; 4,596,556; 4,790,824; 4,941,880; 4,940,460; and PCT publications WO 97/37705 and WO 97/13537. Ballistic powder/particle delivery devices which use compressed gas to accelerate vaccine in powder form through the outer layers of the skin to the dermis are suitable. Alternatively or additionally, conventional syringes may be used in the classical mantoux method of intradermal administration.

Formulations suitable for topical administration include, but are not limited to, liquid and/or semi liquid preparations such as liniments, lotions, oil in water and/or water in oil emulsions such as creams, ointments and/or pastes, and/or solutions and/or suspensions. Topically-administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

A pharmaceutical composition of the invention may be prepared, packaged, and/or sold in a formulation suitable for pulmonary administration via the buccal cavity. Such a formulation may comprise dry particles which comprise the active ingredient and which have a diameter in the range from about 0.5 nm to about 7 nm or from about 1 nm to about 6 nm. Such compositions are conveniently in the form of dry powders for administration using a device comprising a dry powder reservoir to which a stream of propellant may be directed to disperse the powder and/or using a self propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nm and at least 95% of the particles by number have a diameter less than 7 nm. Alternatively, at least 95% of the particles by weight have a diameter greater than 1 nm and at least 90% of the particles by number have a diameter less than 6 nm. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50% to 99.9% (w/w) of the composition, and the active ingredient may constitute 0.1% to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions of the invention formulated for pulmonary delivery may provide the active ingredient in the form of droplets of a solution and/or suspension. Such formulations may be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. The droplets provided by this route of administration may have an average diameter in the range from about 0.1 nm to about 200 nm.

The formulations described herein as being useful for pulmonary delivery are useful for intranasal delivery of an inventive pharmaceutical composition. Another formulation suitable for intranasal administration is a coarse powder comprising the active ingredient and having an average particle from about 0.2 μm to 500 μm. Such a formulation is administered in the manner in which snuff is taken, i.e. by rapid inhalation through the nasal passage from a container of the powder held close to the nose.

Formulations suitable for nasal administration may, for example, comprise from about as little as 0.1% (w/w) and as much as 100% (w/w) of the active ingredient, and may comprise one or more of the additional ingredients described herein. A pharmaceutical composition of the invention may be prepared, packaged, and/or sold in a formulation suitable for buccal administration. Such formulations may, for example, be in the form of tablets and/or lozenges made using conventional methods, and may, for example, 0.1% to 20% (w/w) active ingredient, the balance comprising an orally dissolvable and/or degradable composition and, optionally, one or more of the additional ingredients described herein. Alternately, formulations suitable for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 nm to about 200 nM, and may further comprise one or more of the additional ingredients described herein.

A pharmaceutical composition of the invention may be prepared, packaged, and/or sold in a formulation suitable for ophthalmic administration. Such formulations may, for example, be in the form of eye drops including, for example, a 0.1/1.0% (w/w) solution and/or suspension of the active ingredient in an aqueous or oily liquid carrier. Such drops may further comprise buffering agents, salts, and/or one or more other of the additional ingredients described herein. Other opthalmically-administrable formulations which are useful include those which comprise the active ingredient in microcrystalline form and/or in a liposomal preparation. Ear drops and/or eye drops are contemplated as being within the scope of this invention.

General considerations in the formulation and/or manufacture of pharmaceutical agents may be found, for example, in Remington: The Science and Practice of Pharmacy 21 t ed., Lippincott Williams & Wilkins, 2005.

### EXEMPLIFICATION

The representative Examples that follow are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. It should further be appreciated that the contents of those cited references are incorporated herein by reference to help illustrate the state of the art.

The following Examples contain important additional information, exemplification and guidance that can be adapted to the practice of this invention in its various embodiments and the equivalents thereof. It will be appreciated, however, that these examples do not limit the invention. Variations of the invention, now known and/or further developed, are considered to fall within the scope of the present invention as described herein and as hereinafter claimed.

### Example 1

### TSACs Comprising Iron Oxide, Biotin/NeutrAvidin, and PEG Are Capable of Highly Specific Triggered Self-Assembly

Example 1 demonstrates proof of principle of the methods, compositions and system using biotin and NeutrAvidin coated iron oxide (Fe₃O₄) nanoparticles. Example 1 demonstrates successful blocking of assembly between two TSACs by adding PEG (e.g., 2000-10,000 kDa PEG, such as 5000 kDa and 10,000 kDa) to the surface of biotinylated nanoparticles. Biotinylated TSACs without added PEG demonstrate rapid self-assembly. Example 1 demonstrates that synthesized biotinylated TSACs with PEG tethered by an MMP-2 cleavable peptide substrate have shown an increase in the rate of TSAC assembly by addition of MMP-2.

### Materials and Methods

Synthesis of Nanoparticle Probes

Protease-triggered, self-assembling nanoparticles (i.e. TSACs) were synthesized using 50 nm amine-functionalized, dextran-coated iron-oxide nanoparticles (6.25 pmol/mg Fe), sized by analytical ultracentrifugation (Micromod, Germany). All peptides were obtained at >90% purity (Synpep) and all reagents were obtained from Sigma unless otherwise specified. NeutrAvidin, a commercially available streptavidin, was obtained from Pierce. A high gradient magnetic field filtration column was used between each conjugation (Miltenyi Biotec) and all conjugations were performed at room temperature unless stated. Peptides were synthesized to sequentially contain a lysine (to attach polyethylene glycol polymers to), an MMP-2 cleavage sequence (or scrambled version), and a terminal cysteine (for conjugation onto amines in the dextran coat or lysines on NeutrAvidin proteins). For biotin probes, 1 ml of 0.25 mg/ml N-Succinimidyl 3-[2-pyridyldithio]-propionamido (SPDP) in PBS (0.1 M sodium phosphate, 0.15 M sodium chloride buffer), pH 7.2, was reacted with particle amines (2.5 mg Fe) for 1 hour. Then 1 ml of 1 mg/ml cysteine-containing peptides (acetyl-KGPLGVRGC-X-Biotin) in PBS containing 10 mM EDTA, pH 7.2, was added for 12 hours under N₂ at 4° C. to displace pyridine-2-thione leaving groups. Polyethylene glycol (PEG) polymers with a terminal methoxy cap at one end and 1 ml of 2.5 mM opposing amine-reactive succimidyl α-methylbutanoate (mPEG-SMB, Nektar) in PBS, pH 7.2, was then attached to peptide lysines for 3 hours. NeutrAvidin (Pierce) nanoparticles were formed by modifying particles (2.5 mg Fe) with 1 ml of 0.5 mg/ml biotinamidohexanoyl-6-amino-hexanoic acid N-hydroxy-succinimide ester in PBS, pH 7.2, for 1 hour; and then coated with a saturating concentration of NeutrAvidin (850 μg NeutrAvidin per 2.5 mg nanoparticles) in 5 ml PBS, pH 7.2, for at least 3 hours. The extinction of the solution at 600 nm was measured during incubation to ensure no aggregate formation. Additionally, NeutrAvidin-coated particles were passed through a 0.1 μm filter to confirm mono-dispersity. Using the same conditions described for biotin particle conjugations, peptides (KGPLGVRGC) were conjugated to available lysine amines on NeutrAvidin-coated nanoparticles with SPDP, where mPEG-SMB polymers were conjugated to peptide lysines. Scrambled sequences used for control experiments contained GVRLGPG instead of GPLGVRG.

Extinction, Atomic Force Microscopy, and Magnetic Field Migration Measurements

For all assembly experiments, equimolar ratios of particles were used. All extinction measurements were performed in duplicate in 384 well plates using a SpectraMax Plus spectrophotometer (Molecular Devices, Sunnyvale, Calif.). Biotin and NeutrAvidin probes at 0.5 mg/ml in 0.1 M HEPES, 5 mM calcium chloride, pH 7.2, were mixed at equal ratios and 0.5 μg of the recombinant catalytic domain of matrix metalloproteinase-2 (MMP-2; Biomol) in 6 μl 50 mM Tris, 5 mM calcium chloride, 0.005% Brij-35, pH 7.5, was added to 40 μl probe solution at time zero. For controls, 6 μl of buffer without MMP-2 was added.

The same probe and MMP-2 concentrations were used for Atomic Force Microscopy (AFM) and solution phase magnetic precipitation experiments. AFM measurements were performed using a multimode, Digital Instruments AFM (Santa Barbara, Calif.) operating in tapping mode using FESP Tips (Veeco Nanoprobe™, Santa Barbara, Calif.). AFM reactions were incubated for 3 hours, diluted, and evaporated on freshly-cleaved mica for analysis.

In magnetic precipitation experiments, probe solutions were incubated with or without MMP-2 overnight and placed over a strong magnet for 2.5 minutes.

Magnetic Resonance Imaging (MRI) Detection of Self-Assembly

MRI images were taken on a Bruker 4.7 T magnet, 7 cm bore. Biotin-peptide-PEG and NeutrAvidin-peptide-PEG TSACs were mixed together and serially diluted in 384 well-plate. Serial dilutions of recombinant MMP-2 in 6 μl of Tris buffer were added to each well. After 3 hours, a Carr-Purcell-Meiboom-Gill (CPMG) sequence of sixteen images with multiples of 10.45 ms echo times and a TR of 5000 ms were acquired. T2 maps were obtained for each well by fitting images on a pixel by pixel basis to the equation y=M*exp(−TE/T2) using MATLAB.

Cell Culture

HT-1080 human fibrosarcoma cells (ATCC CCL-121) were cultured in 24-well plates using Minimum Essential Medium Eagle (Invitrogen) with 10% fetal bovine serum (Invitrogen) and 1% penicillin/streptomycin. For MRI experiments, the media was replaced with serum-free Dubelcco's Modified Eagle Medium (DMEM, Invitrogen) containing 10 pM TSAC concentration. The broad-spectrum MMP-2 inhibitor Galardin (Biomol) was added at a concentration of 25 μM in control cultures. Samples of 40 μl were taken at 5 hours for MRI imaging using the same procedures for T2 mapping described above.

For fluorescent labeling experiments, media was replaced with serum-free DMEM containing 200 pM TSAC concentration and cells were placed over a strong magnet. After 3 hours, the medium was removed and the cells were fixed with 2% paraformaldehyde. The cells were permeabilized with 0.1% Triton-X in PBS and incubated with biotin quantum dots (EM: 605 nm, Quantum Dot Corp). Nuclear staining was performed by incubating with 0.001% Hoescht for 1 minute.

### Results

The binding of biotin and NeutrAvidin coated superparamagnetic Fe₃O₄ TSACs was inhibited with PEG polymers that may be proteolytically removed to initiate assembly by matrix metalloproteinase-2 (MMP-2), a protease correlated with cancer invasion, angiogenesis, and metastasis. The invention demonstrates that MMP-2 initiated assembly amplifies the transverse (T2) relaxation of TSAC solutions in magnetic resonance imaging (MRI), enables magnetic manipulation with external fields, and allows MRI detection of tumor-derived cells that produce the protease. This general approach can enable site-selective immobilization and enhanced image contrast in regions of tumor invasion in vivo.

Assembly and Optimization of TSACs

The synthesis of proteolytically-actuated, self-assembling TSACs involves modifying them to be self-complementary, but rendered latent by protease cleavable elements (FIG. 1B). Briefly, 50 nm dextran-coated Fe₃O₄ nanoparticles, sized by analytical ultracentrifugation (Micromod, Germany), were modified with biotin or NeutrAvidin (Pierce, Rockford, Ill.) to generate two populations of particles. When combined in solution, these particles self-assemble through highly stable biotin-NeutrAvidin interactions. To allow enzymatic control of particle assembly, the nanoparticle surfaces of both populations were modified with a MMP-2 peptide substrate, GPLGVRGC, which serves as an anchor for linear PEG chains. PEG is a highly-mobile, hydrophilic polymer with a large sphere of hydration that has been widely used to deter adsorption of proteins or cells on surfaces and to extend therapeutic circulation times in vivo. Thus, linear PEGs of appropriate lengths would inhibit association of 50 nm nanoparticles but still allow MMP-2 proteases (<9 nm) to cleave peptide linkers.

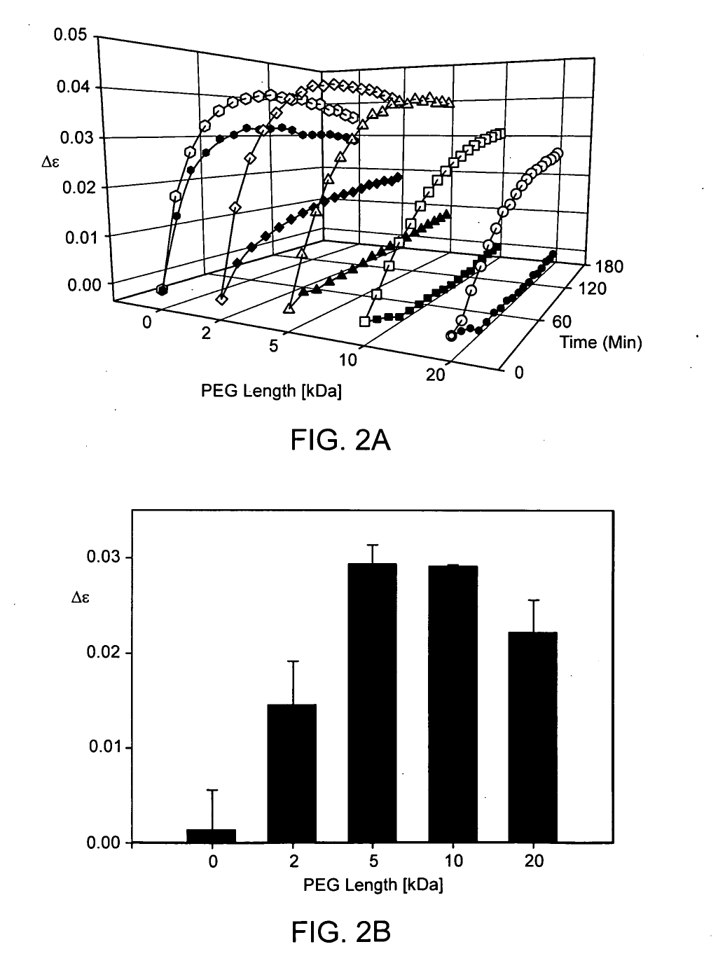

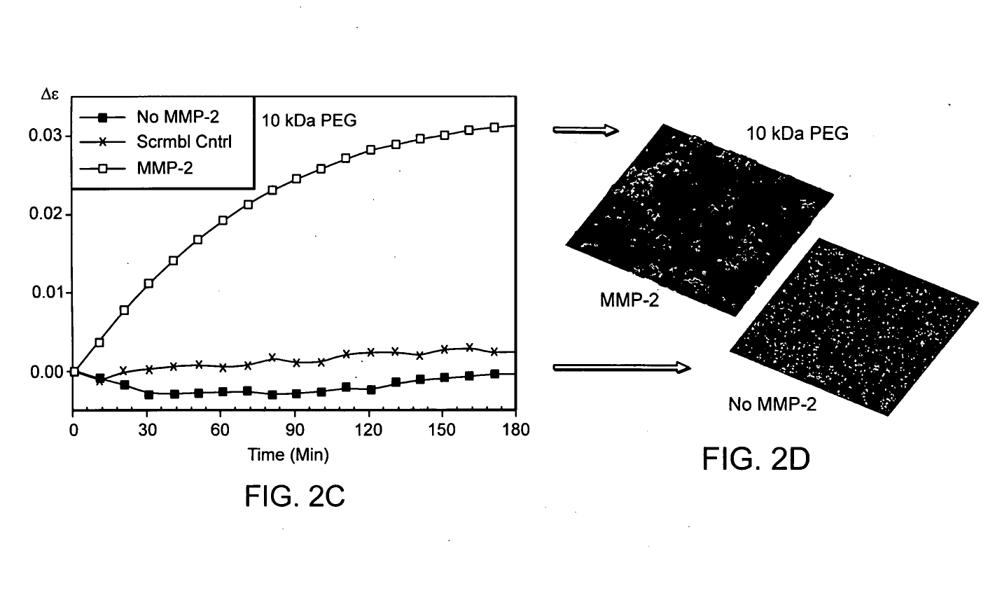

To explore this idea, varying molecular weight PEGs (2, 5, 10, and 20 kDa) were conjugated to biotin and NeutrAvidin particles via MMP-2-cleavable linkers and their ability to assemble with and without MMP-2 tested. The rate and extent of assembly was measured by monitoring changes in the solution extinction at 600 nm (FIG. 2A). Assembly of PEG-coated biotin and NeutrAvidin particles without MMP-2 was found to be inversely related to PEG molecular weight with almost complete inhibition of particle assembly at lengths of 10 kDa or higher. TSACs incubated with MMP-2 also aggregated at a rate inversely related to PEG chain length, likely due to a similar steric repulsion of MMP-2. Comparing the change in extinction of particles incubated with MMP-2 versus those without at 3 hours, the 5 kDa and 10 kDa PEGs allow for higher MMP-2-catalyzed assembly enhancement (FIG. 2B). However, because the 5 kDa PEG cannot completely inhibit particle interaction in their latent state, 10 kDa was chosen as the optimum surface modification for purposes of the experiments described herein.

Release of Peg by MMP-2 is Highly Specific

To further verify that the particle assembly was due to the sequence-specific release of PEG by MMP-2, a scrambled linker with low cleavage-specificity by MMP-2, GPVGLRGC, was generated and conjugated to particles. The TSACs with the scrambled peptide exhibit markedly decreased assembly compared to the specific peptide sequence (FIG. 2C). At 3 hours following MMP-2 addition, assemblies of TSACs with specific MMP-2 substrates, examined by AFM, are as large as 0.5 μm-1 μm, suggesting assembly of tens to hundreds of particles. The TSACs that are not incubated with MMP-2 remain disperse with diameter of approximately 75 nm (FIG. 2D).

Detection of Emergent Properties by MRI

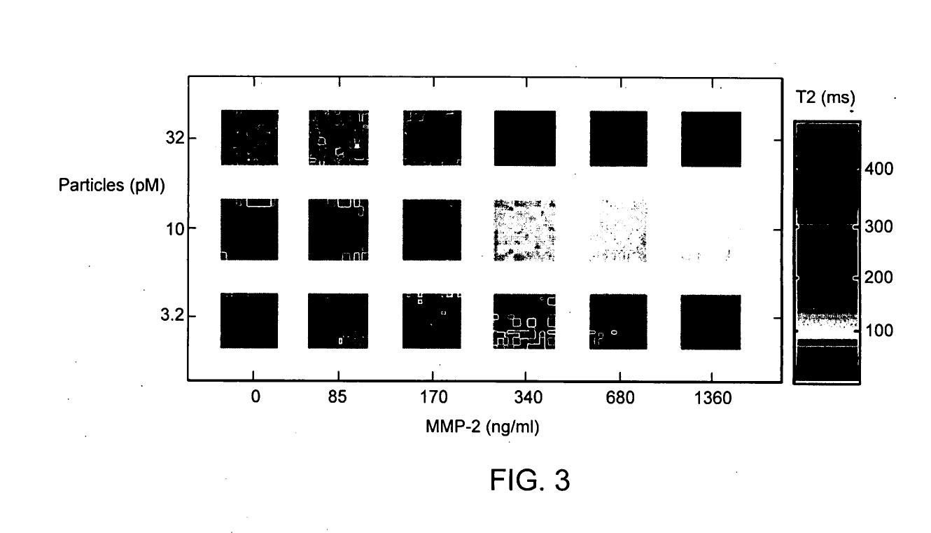

Nanoassemblies of iron oxide particles that form upon proteolytic-activation acquire emergent magnetic properties that may be remotely detected with MRI. The coordination of superparamagnetic Fe₃O₄ magnetic dipoles in assembled TSACs amplifies the diffusional dephasing of surrounding water molecules, causing shortening of T2 relaxation times in MRI. The invention demonstrates measurement of T2 changes allows sensitive, remote detection of protease-triggered assembly across a ten-fold variation in particle concentration (FIG. 3). The concentrations used correspond to 0.7 mg-7.0 mg Fe/kg of solution, spanning the working concentrations typically utilized for tumor and lymphatic targeting in vivo (2.6 mg iron/kg body weight).

TSAC solutions were incubated with varying concentrations of MMP-2 in a 384 well-plate, and their T2 relaxation times were mapped using a Carr-Purcell-Meiboom-Gill (CPMG) sequence on a 4.7 T Bruker MRI. T2 shifts of greater than 150 ms are observed by MMP-2-triggered assembly in a 3.2 pM TSAC solution. For 10 pM and 32 pM concentrations, a T2 shortening approximately 50% of the starting value is observed after incubation with MMP-2. TSACs at a 10 pM concentration were sensitive to at least 170 ng/ml (9.4 U/ml) of MMP-2, which compares favorably with levels found in tumor tissue of MMP-2 expressing cancer cells (435 U MMP-2/g).

TSACs in Cell Culture Assays

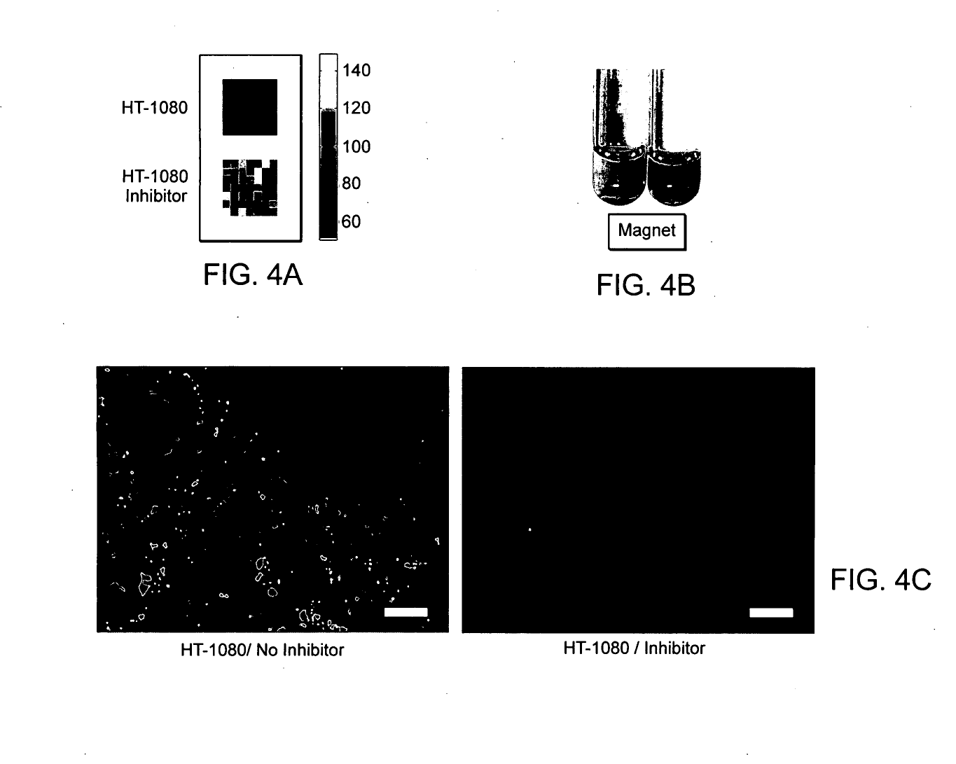

Next, the utility of the protease-triggered TSACs was explored in complex biological specimens where non-specific protein adsorption is often problematic. Specifically, latent TSACs were incubated in cell culture medium above living human fibrosarcoma cells, HT-1080s, which constitutively express and activate MMP-2. MMP-2 is a zinc binding protease with cleavage specificity for Type IV collagen, the principal constituent of basement membranes. Upregulation of MMP-2 activity leads to invasive proliferation and metastases of cancer cells by breaking down tissue barriers. TSACs (10 pM) were incubated over HT-1080 cells for 5 hours and T2 maps of media samples were generated with MRI. A substantial shortening in T2 was detected in the media over HT-1080 cells versus media over cells incubated with the broad-spectrum MMP inhibitor Galardin (FIG. 4A).

Triggered assembly of the TSACs can also be used to magnetically target nanoassemblies to cells. Similar to the T2 relaxivity enhancement in MRI, as the magnetic domains of coalesced TSACs coordinate to form an amplified cumulative dipole, they become more susceptible to long-range dipolar forces. This phenomenon allows manipulation of the nanoassemblies with imposed magnetic fields, while isolated particles remain unaffected. Using a high-gradient permanent magnet, MMP-2 triggered assemblies of 1.5 nM iron oxide particles can be visually drawn out of solution, while non-activated particles remain disperse (FIG. 4B). To demonstrate that this can be extended towards targeting particles onto cancer cells, HT-1080 cultures were placed over a strong permanent magnet and incubated with TSACs at a 150 pM concentration. After 3 hours, the medium was removed and the cells were washed, fixed, and stained for aggregates using a biotinylated fluorescent probe. Bright fluorescent staining of particle assemblies is seen over HT-1080 cells, while weak diffuse staining, indicating little to no targeting, is seen over cells incubated with the inhibitor Galardin (FIG. 4C).

### Discussion

This disclosure represents the first demonstration of protease-triggered TSAC self-assembly. This system differs from the reported use of enzymatic cleavage to prevent assembly; rather it exploits proteolytic activity to construct multimeric assemblies with emergent properties. Data have also been obtained that demonstrates that peptide-modified semiconductor quantum dots could precisely target tumors in whole animals and subcellular organelles in living cells. This disclosure extends the ability of TSACs not only to target sites of interest, but to interact with the processes of disease by harnessing biological machinery to assemble nanomaterials with amplified properties. The disclosure shows that polymeric protection can temporarily shield dissimilar complementary ligands, including both small molecules (biotin) and tetrameric proteins (NeutrAvidin). Accordingly, in contrast to recent reports of proteolytic activation of cell-penetrating peptides and peroxidase-initiated TSAC assembly, this approach can be considered entirely modular and thereby generalizable whereby key features (e.g. biochemical trigger, molecular recognition) may be altered without significant re-engineering. Formulations with new functionalities could be easily developed by substituting the complementary binding pairs, cleavable substrates (e.g. glycans, lipids, oligonucleotides), or multivalent nanoparticle cores (e.g. gold, quantum dot, dendrimer) to extend the capabilities of existing modalities.

### Example 2

### TSAC Self-Assembly Directed by Antagonistic Kinase and Phosphatase Activities

### Introduction

Example 2 demonstrates a TSAN used to dynamically report the activity of a prototypical antagonistic enzyme pair (tyrosine kinase and phosphatase) via T2 relaxation changes in magnetic resonance imaging (MRI). MRI, which is widely used in medicine, provides exquisite 3-D anatomical detail with relaxation acquisition timescales on par with many intracellular enzyme processes (Shapiro et al., 2006, Magn. Reson. Imaging, 24:449). The TSAN of Example 2 leverages the spin-spin (T2) relaxation enhancement upon superparamagnetic TSAC self-assembly (Perez et al., 2002, Nat. Biotechnol., 20:816; and Harris et al., 2006, Angew. Chem. Int. Ed. Engl., 45:3161) by coupling TSAC self-assembly to the presence of kinase activity. Kinase-induced nanoassemblies enhance T2 relaxation of hydrogen atoms at picomolar enzyme concentrations and are shown to be reversible by introducing excess phosphatase activity. This system may be optimized to non-invasively report the balance between enzyme activities following delivery into cells and may facilitate new screens for inhibitors in vitro.

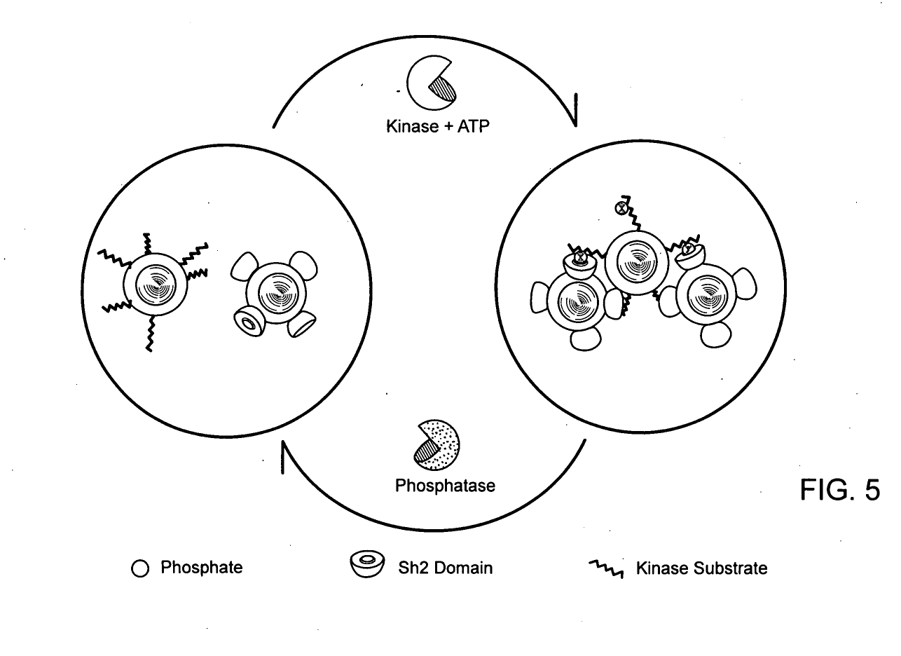

To construct a TSAN comprising TSACs that can self-assemble in the presence of kinase activity and re-disperse in the presence of phosphatase activity, two TSAC populations were synthesized to interact in a coordinated fashion (FIG. 5). The first population was modified with peptide substrates that may be phosphorylated by Abl tyrosine kinase and dephosphorylated by a phosphatase. The second population was modified with Src Homology 2 (SH2) domains that recognize and bind the phosphorylated Abl kinase substrate in a sequence-specific manner. Together, these TSACs process kinase and phosphatase activities by assembling as peptides become phosphorylated and disassembling as phosphates are removed. Magnetic dipoles in TSAC assemblies coordinate and more efficiently dephase hydrogen protons in MRI, allowing T2 relaxation mapping of kinase function. Conceptually, this design is akin to the kinase/phosphatase FRET sensors developed (Sato et al., 2002, Nat. Biotech., 20:287; Wang et al., 2005, Nature, 434:1040; Ting et al., 2001, Proc. Natl. Acad. Sci., USA, 98:15003; and Violin et al., 2003, J. Cell Biol., 161:899) among many other fluorescence-based kinase sensors (Shults et al., 2005, Nat. Methods, 2:277; Prinz et al., 2006, Cell Signal., 18:1616; Rininsland et al., 2004, Proc. Natl. Acad. Sci., USA, 101:15295; and Shults et al., 2003, J. Am. Chem. Soc., 125:14248), but instead of transducing enzyme activities into optical fluorescence changes, activity is encoded via nuclear magnetic resonance (NMR) relaxation changes Perez et al., 2002, Nat. Biotechnol., 20:816; Perez et al., 2004, Chembiochem, 5:261; Atanasijevic et al., 2006, Proc. Natl. Acad. Sci., USA, 103:14707; and Wang et al., 2006, J. Am. Chem. Soc., 128:2214). While nanoparticle-based T2-sensing of analytes and proteases has been demonstrated Perez et al., 2002, Nat. Biotechnol., 20:816; and Harris et al., 2006, Angew Chem. Int. Ed. Engl., 45:3161), the translation of this technology to reversibly sensing multiple enzyme activities has not been accomplished. Recently, two gold nanoparticle-based approaches have sensed either kinase or phosphatase activity in irreversible, two-step assays (Wang et al., 2006, J. Am. Chem. Soc., 128:2214; and Choi et al., 2006, Angew Chem. Int. Ed. Engl., 46:707). These designs provide new avenues for colorimetric screening of enzyme inhibitors, yet lack the capacity to continuously analyze both kinase and phosphatase balance.

### Materials and Methods

### Materials

All chemicals and reagents were purchased from Sigma-Aldrich unless otherwise specified. Plasmid expressing GST-Cys-SH2 was supplied by Dr. Barbara Imperiali (Department of Chemistry, MIT). Peptides were synthesized following standard Fmoc solid phase peptide synthesis method using an ABI Model 433A peptide synthesizer in MIT center for cancer research biopolymer laboratory. Nanoparticle size was measured using Zetasizer (Malvern Instruments). MRI images were taken on a Bruker 4.7 T magnet. All enzyme reactions were carried out at 30° C. unless otherwise specified. Aminated nanoparticles (i.e. TSACs) were synthesized according to published procedures.

### Expression and Purification of SH2 Domain

BL21-Gold(DE3) cells harboring GST-Cys-Crk SH2 plasmid (pGEX4T-Cys-CrkSH2) were grown to midlog phase in LB media containing 50 μg/ml carbenicillin at 37° C., 220 rpm. Protein expression was induced with addition of 0.1 mM IPTG after cells were cooled to 16° C., and then cells were incubated at 16° C. for 21 hours. Cells were centrifuged at 5000 rpm at 4° C. for 30 minutes, and the cell pellet was resuspended in a lysis buffer (1×PBS, 100 mM EDTA, 1% Triton X-100, 10% glycerol, 1 mg/ml lysozyme, 1× protease inhibitor cocktail set III (CalbioChem)) and incubated for 30 minutes at 4° C. After sonication, the soluble fraction was isolated from cell debris after centrifugation for 30 minutes at 14,000 rpm and then purified using glutathione sepharose 4B affinity column (Amersham Biosciences) following the manufacture's protocol. Eluted proteins were dialyzed with 7 kDa molecular weight cutoff dialysis cassette (Slide-a-Lyzer, Pierce) against 1×PBS and characterized by SDS-PAGE. To remove the GST tag, 1 mg/ml protein was treated with 50 U/ml TEV protease (Invitrogen) in a TEV protease buffer (50 mM Tris-HCl, 0.5 mM EDTA, pH 8.0) in the presence of 1 mM DTT. After a 4 hour incubation at 25° C., the cleavage reaction mixture was subject to a glutathione column and then a Ni⁺²-NTA column to sequentially remove cleaved GST tag and TEV protease, respectively. To ensure that cysteine thiols of cys-SH2 domain were fully reduced, cys-SH2 domain was passed through reducing column (Reduce-Imm Immobilized Reductant Column, Pierce) following manufacture's instructions immediately prior to nanoparticle conjugation.

### Preparation of Peptide-Presenting TSACs and SH2-Conjugated TSACs

Maleimide-activated TSACs were prepared by conjugating NHS-PEO12-maleimide (succinimidyl-[(N-maleimidopropionamido)-dodecaethyleneglycol]ester, Pierce) to aminated nanoparticles (i.e., aminated TSACs). Typically, 0.25 mg Fe nanoparticles were incubated with 4 mM of NHS-PEO12-maleimide for 30 minutes at 25° C. and then purified using a magnetic field filtration column (Miltenyi Biotec). SH2 conjugated particles were prepared by incubating 1 mg/ml Cys-SH2 with maleimide presented nanoparticles (0.25 mg Fe) for 3 hours at room temperature. Unreacted cys-SH2 domain was removed using a magnetic field filtration column. Peptides were conjugated by activating amine-nanoparticles with NHS-PEO12-maleimide as above, followed by addition of peptide substrate. Particles were filtered for 2 hours after peptide addition. The peptides used in this investigation were synthesized as follows:

(Ahx: Aminohexanoic Acid)

CRK SH2-Binding:

TAMRA-C(Ahx)QpYDHPNI-CONH₂
TAMRA-C(Ahx)QYDHPNI-CONH₂

Non-Binding Abl Substrate:

TAMRA-(Ahx)EAIpYAAPFAKKKC-CONH₂

CRK SH2-Binding Abl Substrates:

TAMRA-(Ahx)SRVGEEEHVpYSFPNKQKSAEC-CONH₂
TAMRA-(Ahx)SRVGEEEHVYSFPNKQKSAEC-CONH₂
TAMRA-(Ahx)SRVGEEEHVFSFPNKQKSAEC-CONH₂

### SH2-Binding Peptide-Mediated TSAC Assembly

Nanoparticles (i.e. TSACs) presenting Crk SH2-binding peptide (either phosphorylated or unphosphorylated) or non-binding Abl substrate (phosphorylated) were incubated with Crk-SH2 TSACs at 10 μg Fe/ml (12 nM TSAC concentration) and monitored with DLS over time.

### Kinase-Directed TSAC Assembly

TSACs presenting 10 μg Fe/ml kinase substrate peptide (12 nM TSAC concentration) and 10 μg Fe/ml SH2-presented TSACs (12 nM TSAC concentration) were mixed in a kinase reaction buffer (20 mM Tris-HCl, pH 7.5, 2 mM MgCl₂, 20 mM NaCl, 0.2 mM EGTA, 0.4 mM DTT, 0.004% Brij 35, 0.2 mM ATP) in a total volume of 50 μl. Kinase reaction was initiated by adding indicated amount of Abl kinase (New England Biolabs). TSAC assemblies were characterized by DLS over time or MRI.

### Phosphatase-Directed TSAC Disassembly

5 μg Fe/ml SH2 TSACs (6 nM TSAC concentration) were added to 5 μg Fe/ml phosphorylated tyrosine containing peptide TSACs (6 nM TSAC concentration) in a buffer solution (20 mM Tris-HCl pH 7.5, 20 mM NaCl, 0.4 mM Na₂EDTA, 2 mM DTT, 0.004% Brij 35) to initiate TSAC assembly. YOP protein tyrosine phosphatase (New England Biolabs, 2 U/μl) was added when size of assembled TSACs reached to about 400 nm in radius.

### Reversal of Kinase Induced TSAC Assembly by Phosphatase

TSAC assembly was initiated following same protocols described above. Then, 5 U/μl YOP phosphatase was directly added into a kinase reaction mixture. Size measurement was restarted right after thoroughly mixing the reaction mixture.

### MRI Imaging of TSACs

All TSAC solutions were prepared in final concentration of 10 μg Fe/ml (12 nM TSAC concentration) in 70 μl of kinase reaction buffer. TSAC mixtures were incubated at 30° C. for 3 hours after kinase additions (0, 0.05, 0.1, 0.2, 0.5 U/μl), and then MRI images were taken using a 4.7 T Bruker magnet (7 cm bore) using T2-mapping Carr-Purcell-Meiboom-Gill (CPMG) pulse sequence. To reverse assembly, 4 U/μl YOP phosphatase or 0.1 mM free pY-peptide was added to an assembled TSAC solution containing 0.2 U/μl Abl kinase. The MRI image was taken after 10 minutes at room temperature.

### Results

### Phosphopeptide-SH2 Domain Binding can Trigger Self-Assembly.

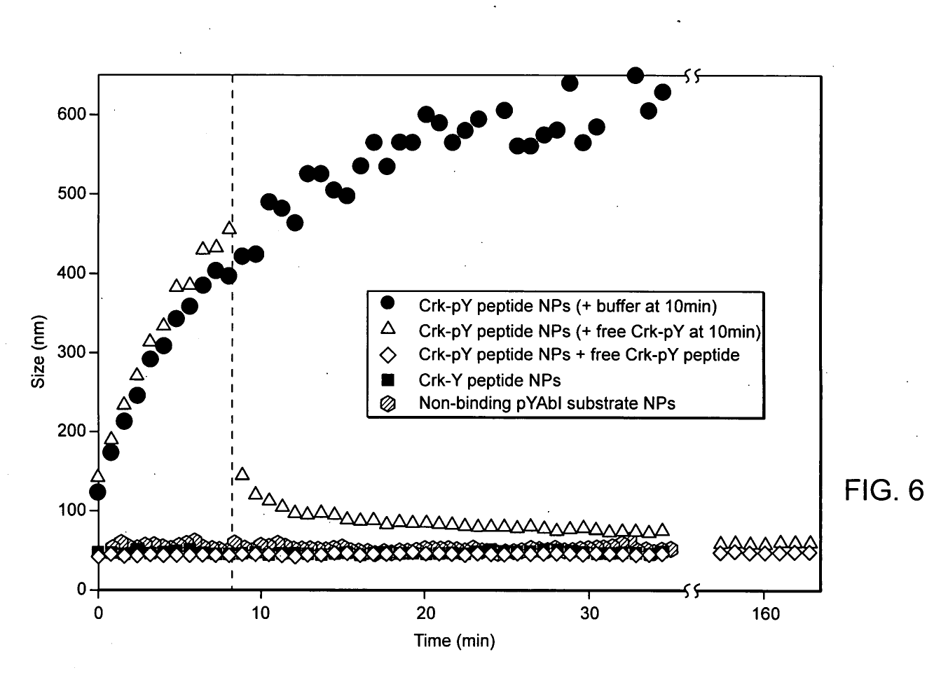

Dextran-coated iron oxide TSACs were synthesized, cross-linked, and aminated according to published procedures (Palmacci et al. 1993, U.S. Patent Vol. 5, p. 176; Shen et al., 1993, Magn. Reson. Med., 29:599; and Josephson et al., 1999, Bioconjug. Chem. 10:186). The Crk SH2 domain was genetically modified to contain an N-terminal cysteine to allow convenient conjugation to TSACs. GST-tagged cysteine-SH2 was expressed in bacteria, purified, and the GST affinity label was removed. Reduced cysteine-SH2 was conjugated to amine-TSACs via highly flexible heterobifunctional linkers, each containing 12 polyethylene oxide units (54.4 Å), to increase conformational freedom. In parallel, a phosphotyrosine (pY) sequence with low μM binding affinity to Crk SH2 (-QpYDHPNI-) (Songyang et al., 1993, Cell 72:767; and Vazquez et al., 2005, J. Am. Chem. Soc., 127:1300) was synthesized with an N-terminal cysteine and attached to a second population of TSACs using the same linker. Even at TSAC concentrations three orders of magnitude lower than the free peptide affinity (12 nM TSACs), these TSACs rapidly assembled when combined, as shown by the 10-fold hydrodynamic radius increase within 15 minutes using dynamic light scattering (DLS) (FIG. 6). In the presence of 200 μM free pY peptide, assembly was inhibited. Further, SH2-TSACs were able to discriminate pY-TSACs from Y-TSACs (unphosphorylated tyrosine) and from a phosphopeptide not expected to bind to CRK SH2 (EAIpYAAPFAKKKC) (Songyang et al., 1993, Cell 72:767).

To test the reversibility of this system, pY TSAC and SH2 TSAC self-assembly was interrupted with addition of 200 μM free pY-peptide or 2 μl of buffer (FIG. 6). While mixing shear stress had no affect on TSAC assembly, particles with 200 μM free peptide rapidly disassembled, dispersing over time. These data demonstrate that phosphopeptide-SH2 domain binding can efficiently induce assembly at TSAC concentrations relevant to MRI.

### Phospho-Dependent TSAC Assembly Effectively Monitors Kinase Activity

The rapid association of pY-TSACs with SH2-TSACs indicated that phospho-dependent TSAC assembly may provide a rapid mechanism for probing kinase activity.

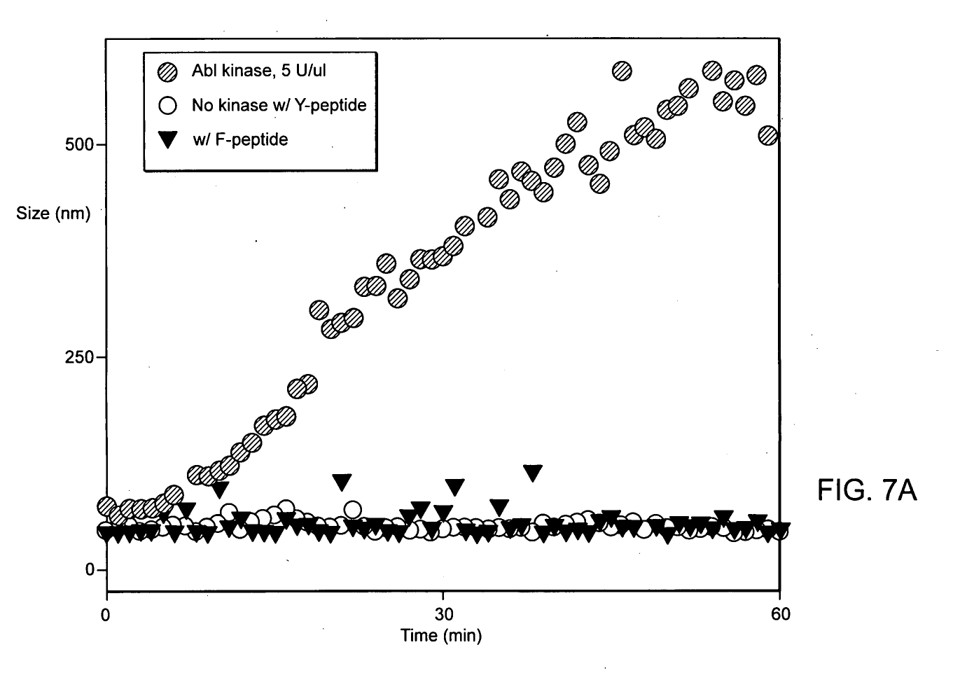

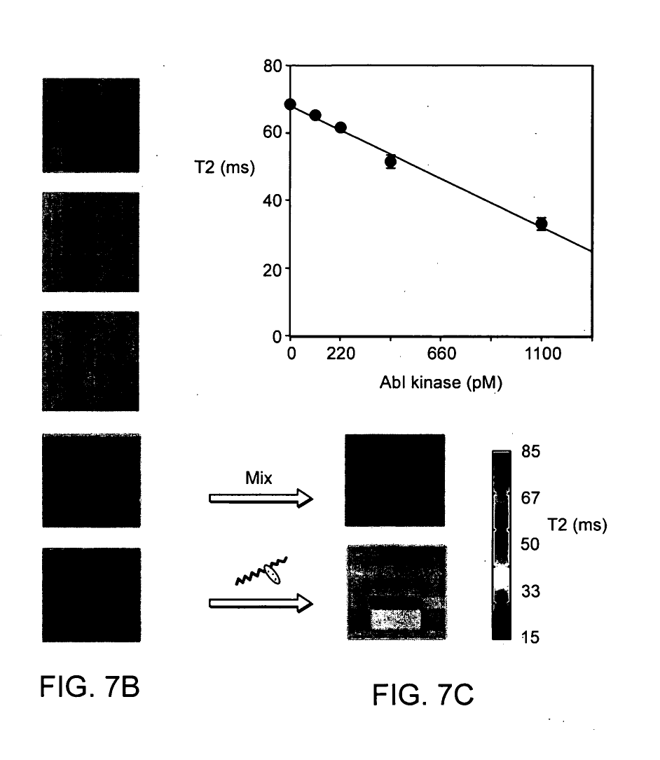

To begin, a kinase substrate (SRVGEEEHVYSFPNKQKSAEC) derived from paxillin was chosen for its Crk SH2 binding and specificity to Abl (Bellis et al., 1995, J. Biol. Chem., 270:17437; and Schaller et al., 1995, Mol. Cell. Biol., 15:2635). Three versions of this peptide substrate were synthesized: a phosphorylated substrate (pY-Abl), an unphosphorylated substrate (Y-Abl), and a substrate in which the receptor tyrosine was replaced with a phenylalanine (F-Abl). Abl kinase rapidly directed assembly in solutions containing Y-Abl TSACs with SH2 TSACs, while F-Abl peptide control remained dispersed in DLS (FIG. 7A). Using a 4.7 T Bruker MRI magnet, the ability of TSAC self-assembly to transduce kinase activity into NMR T2 relaxation changes was determined (FIGS. 7B,C). Quantifiable T2 relaxation enhancements in solutions containing Y-Abl TSACs with SH2 TSACs were observed in the presence of as little as 11 fentomoles of added kinase (110 pM kinase concentrations=0.05 U/μl; FIG. 7C). Further, T2 enhancement was lost upon addition of free pY-Abl substrate, demonstrating that kinase-directed TSAC assembly depended on phosphopeptide-SH2 domain interactions that were reversible by competition (FIG. 7B).

### Phosphatase Activity Opposes Kinase-Directed Self-Assembly

As TSACs aggregate, tyrosine-linked phosphates become sequestered in SH2 domain binding pockets. Having demonstrated that addition of free pY-peptide was able to reverse TSAC binding, it was then determined that phosphatase activity could oppose kinase-directed self-assembly by removing phosphates from tyrosine residues.

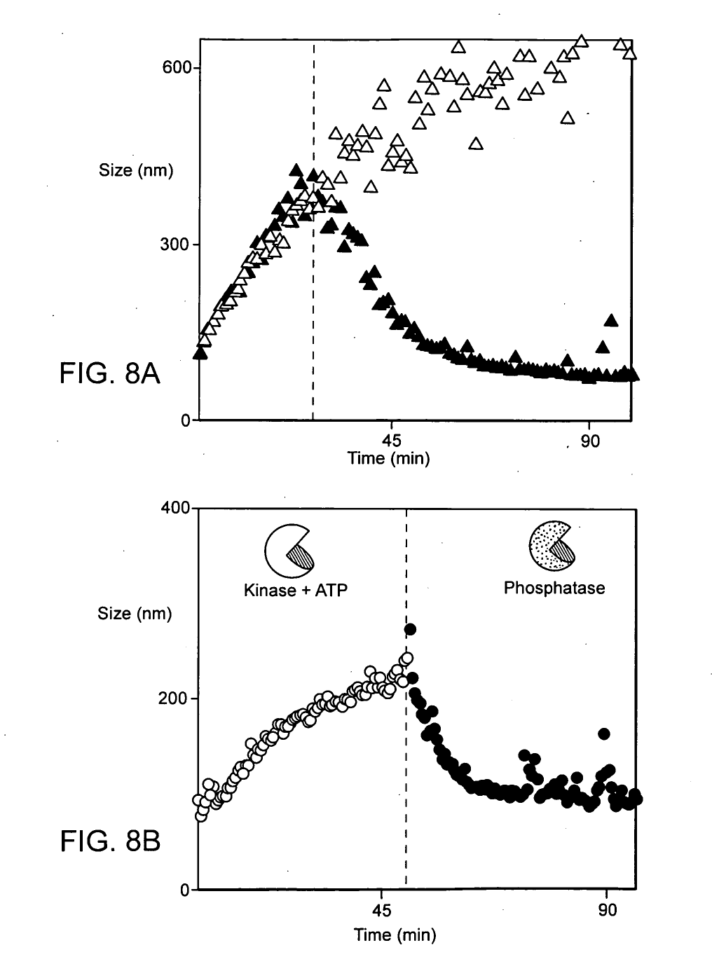

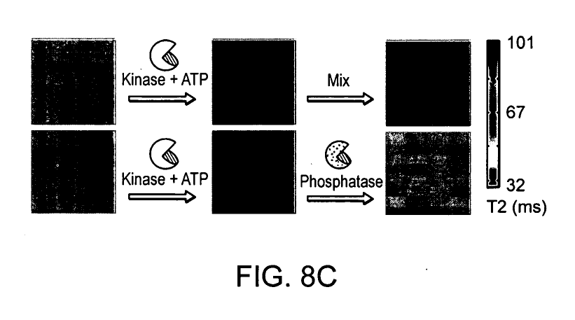

To begin, the ability of YOP phosphatase ability to counteract the rapid association of pY-Abl TSACs with SH2 TSACs was tested. TSAC nanoassemblies with hydrodynamic radii of approximately 400 nm were allowed to form, at which point, phosphatase or buffer was added (FIG. 8A). In the presence of phosphatase, TSACs rapidly disassociate, eventually re-dispersing in solution.

Next, the potential for kinase and phosphatase to sequentially control TSAC assembly was determined. Y-Abl TSACs and SH2 TSACs were first exposed to kinase activity and subsequently to an excess of antagonistic phosphatase activity. Indeed, kinase-catalyzed TSAC assembly was efficiently reversed by addition of excess phosphatase (FIGS. 4B,C), illustrating the potential for this system as a reversible magnetic resonance (MR) sensor of cycling kinase/phophatase activities.

### Discussion

These results demonstrate that phosphatase is able to halt TSAC assembly, by removing phosphates from free TSACs, and also to deconstruct phospho-dependent nanoassemblies, by removing phosphates as they dynamically disassociated with SH2 domains. The present invention encompasses the recognition that rapid reversal of TSAC assembly, along with the enhancement of TSAC avidity over anticipated monovalent binding (assembling at TSAC concentrations 1000-fold below peptide/SH2 affinities) are indications of polyvalent TSAC binding (Mammen et al., 1998, Angewandte Chemie-International Edition, 37:2755). Unlike monovalent interactions, the disassociation rate of polyvalent species may be accelerated by the presence of monomeric competitor (Rao et al., 1998, Science, 280:708). Synthetically, polyvalency has been exploited to develop improved biological inhibitors (Mammen et al., 1998, Angewandte Chemie-International Edition, 37:2755), targeting agents (Weissleder et al., 2005, Nat. Biotechnol., 23:1418; and Simberg et al., 2007, Proc. Natl. Acad. Sci., USA, 104:932) and affinity chromatography procedures (Rao et al., 1998, Science, 280:708). Here, polyvalent binding was exploited to engineer a reversible TSAC system that forms assembles of highly stable nanostructures, yet may also be rapidly disassembled by competition.

Design and synthesis of a TSAC system that processes two antagonistic enzymes inputs (tyrosine kinase/phosphatase) to output enhanced T2 relaxation in the presence of net kinase activity is demonstrated. Phosphopeptide-directed assembly occurred within minutes, enabling a rapid MR visualization of kinase activity at nanomolar TSAC and picomolar kinase concentrations. Looking forward, as MRI field strengths increase and methods for labeling cells with nanomaterials advance, optimizations of this design may enable MRI mapping of cytosolic enzyme activity in optically opaque media and in vivo.

### Example 3

### TSAC Self-Assembly Gated by Logical Proteolytic Triggers

### Introduction

Emergent electromagnetic properties of nanoparticle self-assemblies are being harnessed to build new medical and biochemical assays with unprecedented sensitivity. Nanoparticle assembly has been exploited to probe for a host of pathological inputs in vitro, including DNA (Perez et al., 2002, Nat. Biotechnol., 20:816; and Mirkin et al., 1997, Science, 277:1078), RNA (Perez et al., 2002, Nat. Biotechnol., 20:816), proteins (Georganopoulou et al., 2005, Proc. Natl. Acad. Sci., USA, 102:2273; and Perez et al., 2004, Nano Letters, 4:119), viruses (Perez et al., 2003, J. Am. Chem. Soc., 125:10192), and enzymatic activity (Perez et al., 2004, Nano Letters, 4:119; Harris et al., 2006, Angew Chem. Int. Ed. Engl., 45:3161; and Wang et al., 2003, Angew Chem. Int. Ed., 42:1375). Typically, nanoparticle systems are designed to sense single molecular targets. While this methodology has been effective for in vitro applications, the future development of highly diagnostic in vivo sensors may benefit from the ability to monitor multiple aspects of disease. In this report we describe a method whereby inorganic nanocrystals (i.e. TSACS) may utilize Boolean logic to simultaneous process two inputs associated with cancer invasion (MMP-2 and MMP-7). Disperse, superparamagnetic Fe₃O₄ TSACs are designed to coalesce in response to logical “AND” or “OR” functions. In either system, TSAC self-assembly amplifies the T2 relaxation of hydrogen protons, enabling remote, MRI-based detection of logical function. The present invention encompasses the recognition that, in the future, these sensors may be optimized to monitor a diversity of logical inputs both in vitro and in vivo.

### Materials and Methods

### Production of TSACs

Unless otherwise stated all reagents were purchased from Sigma-Aldrich and all reactions were performed at room temperature. Superparamagnetic iron oxide nanoparticles were synthesized according to the published protocol. Briefly, dextran-coated iron oxide nanoparticles were synthesized, purified, and subsequently cross-linked using epichlorohydrin. After exhaustive dialysis, particles were aminated by adding 1:10 v/v ammonium hydroxide (30%) and incubated on a shaker overnight. Aminated-nanoparticles were subsequently purified from excess ammonia using a Sephadex G-50 column and concentrated using a high-gradient magnetic-field filtration column (Miltenyi Biotec).

### Peptide-Polymer Synthesis

Peptides were synthesized in the MIT Biopolymers core to sequentially contain a lysine (for the attachment of polyethylene glycol polymers), a MMP-cleavage sequence, and a terminal cysteine (for conjugation onto amines in the dextran coat or lysines on NeutrAvidin (Pierce) proteins. Peptide purity was verified with HPLC and mass spectrometry. Amine-reactive 20 kDa mPEG-SMB reagents (methoxy-polyethylene glycol-succimidyl α methylbutanoate) were purchased from Nektar Therapeutics. The following sequences were used in this investigation: (N->C) MMP-2 substrate: G-K(TAMRA)-G-P-L-G-V-R-G-C-CONH2; MMP-7 substrate: G-K(TAMRA)-G-V-P-L-S-L-T-M-G-C-CONH2; MMP-7-MMP-2 tandem substrate: TAMRA-G-K-G-V-P-L-S-L-T-M-Ahx-G-P-L-G-V-R-G-C-CONH2 where K(TAMRA)=Lys(DDE) substituted with 5(6)-TAMRA, TAMRA=5(6)-TAMRA, and Ahx=aminohexanoic acid. Peptides were reacted with polymers in PBS+0.005 M EDTA pH 7.2 at 500 μM and 400 μM, respectively, for at least 24 hours with shaking. Free peptide was removed by reducing with 0.1 M TCEP and filtering using a G-50 Sepahadex column. Reduced polymer was then quantified using fluorochrome extinction and added to TSAC preparations as described below.

### Ligand TSAC Synthesis

Following each conjugation, TSACs were purified using a high-gradient magnetic-field filtration column (Miltenyi Biotec). Aminated nanoparticles (1 mg Fe/ml) were simultaneously reacted with biotinamidohexanoyl-6-aminohexanoic acid N-hydroxysuccinimide ester and 4-Maleimidobutyric acid N-hydroxysuccinimide ester (0.8 mM and 1.2 mM, respectively) in 0.1 M HEPES, 0.15 M NaCl, pH 7.2 buffer for 30 minutes. Purified nanoparticles (1 mg Fe/ml) were then combined with reduced peptide-polymers (1 mM) in phospho-buffered saline+0.005 M EDTA, pH 7.2 and incubated for at least 2 hours. Particles were again purified and used in subsequent assembly experiments.

### Receptor TSAC Synthesis

Aminated nanoparticles (1 mg Fe/ml) were reacted with biotinamidohexanoyl-6-aminohexanoic acid N-hydroxysuccinimide ester (0.03 mM) in 0.1 M HEPES, 0.15 M NaCl, pH 7.2 buffer for 30 minutes. Following filtration, nanoparticles (1 mg Fe/ml) were combined with a saturating concentration of NeutrAvidin protein (Pierce, 5 mg/ml) and incubated for at least 3 hours. The extinction of nanoparticle solutions at 600 nm was monitored during NeutrAvidin-coating to ensure cross-linking was not occurring. After purification, NeutrAvidin particles were passed through a 0.2μ filter to ensure removal of any aggregates. NeutrAvidin nanoparticles (1 mg Fe/ml) were then reacted with 2 mM 4-Maleimidobutyric acid N-hydroxysuccinimide ester for 30 minutes, purified, and incubated with 1 mM peptide-polymers for at least 2 hours as before. Particles were finally purified from excess peptide-polymer and used in subsequent assembly experiments.

### Dynamic Light Scattering Studies

All dynamic light scattering experiments were performed in 100 μl solutions of 0.1 M HEPES, 0.15 M NaCl, 0.005 M CaCl₂ at 25° C. with TSACs at 40 μg Fe/ml (added at equimolar concentrations). To begin an experiment, catalytic domains of MMP-2 and MMP-7 (Biomol) were added in 5 μl to 95 μl of TSACs or 5 μl control buffer was added. Kinetic dynamic light scattering intensity size measurements were taken using a Malvern ZS90 and hydrodynamic radius was plotted vs time.

### MRI Detection of TSAC Self-Assembly

MRI T2 mapping was performed using a 7 cm bore, Bruker 4.7 T magnet. TSACs were mixed together in 384-well plate to contain 95 μl total sample/well. Recombinant MMP-2 or MMP-7 (Biomol) was pre-incubated at 37° C. for 30 minutes to activate and added in a total of 5 μl 50 mM Tris-HCl, 5 mM CaCl₂, 0.005% Brij-35, pH 7.5 were added to each well. After a 3 hour incubation, T2 relaxation maps were obtained. Data in each well were displayed by fitting images on a pixel by pixel basis to the equation y=M*L10^{(-TE/T2)} using MATLAB.

### Results

### Design and Synthesis of TSACs: General Considerations

Logical operations were designed to analyze inputs of two matrix-metalloproteinases (MMPs), a family of at least 26 members of secreted and membrane bound proteases that have been studied extensively for their role in cancer (Chang et al., 1998, Nature, 394:527). In particular, matrix-metalloproteinase-2 (MMP-2), is over-expressed in many cancers, including breast cancers, and is an indicator of cancer invasiveness, metastasis, angiogenesis, and treatment efficacy (Stearns et al., 1993, Cancer Res., 53:878; Talvensaari-Mattila et al., 2003, Brit. J Cancer, 89:1270; Davidson et al., 1999, Gynecol. Oncol., 73:372; Kanayama et al., 1998, Cancer, 82:1359; Fang et al., 2000, Proc. Natl. Acad. Sci., USA, 97:3884; Ratnikov et al., 2002, Lab. Invest., 82:1583; and Giannelli et al., 1997, Science, 277:225). MMP-7, a protease with broader substrate specificity, is thought to facilitate early stages of mammary carcinoma progression (Rudolph-Owen et al., 1998, Cancer Res., 58:5500; and Hulboy et al., 2004, Oncol. Rep., 12:13). In tissues excised from breast cancer patients, both MMP-2 and MMP-7 were expressed at statistically higher levels in carcinogenic than in normal breast tissues (Pacheco et al., 1998, Clin. Exp. Metastasis, 16:577), highlighting their potential utility as dual markers of neoplastic inception. The present invention encompasses the recognition that, by using dynamic light scattering and MRI, logical sensors can probe samples for the presence of both MMP-2 and MMP-7 (“AND” function) or for the presence of either MMP-2 or MMP-7 (“OR” function).

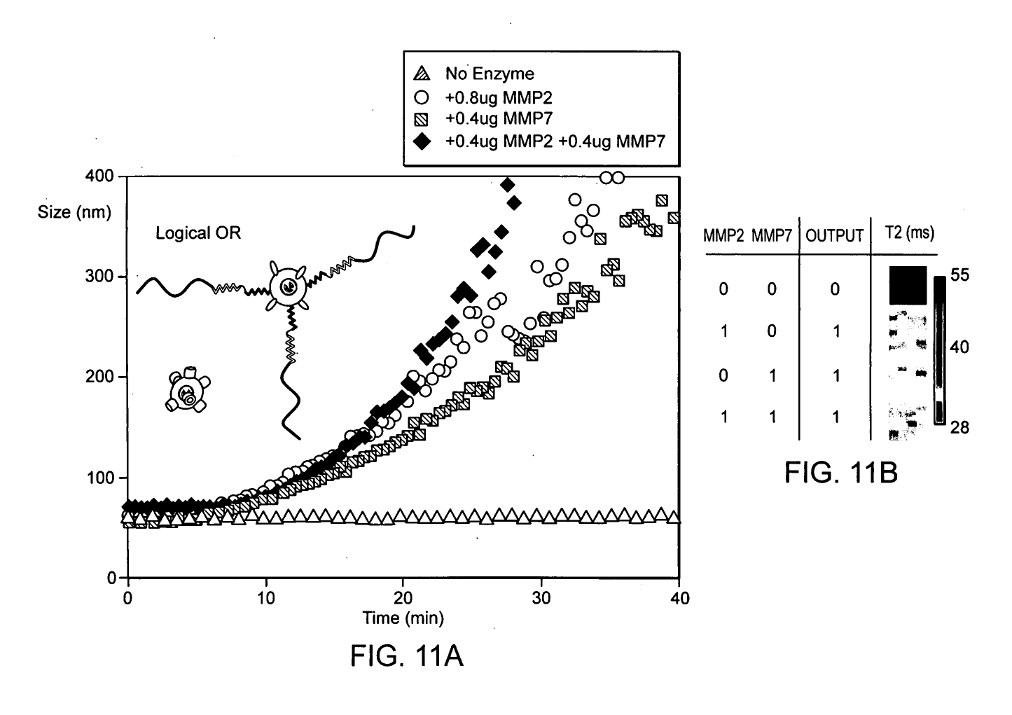

To synthesize both sensor types, two kinds of TSACs were initially engineered: one with a tethered ligand (biotin) and the other with its receptor (NeutrAvidin). These TSACs were stable separately, but aggregated readily when combined. We sought to completely mask these groups by attachment of peptide-polyethyleneglycol (PEG) conjugates to conditionally prevent assembly. Previously, we demonstrated that two 10 kDa PEG-modified TSACs could mutually deter each other's binding (Harris et al., 2006, Angew Chem. Int. Ed. Engl., 45:3161). Here, by extending the polymer length to 20 kDa, we demonstrate that modification of only one TSAC can completely inhibit the binding of an unmodified TSAC (FIG. 11).

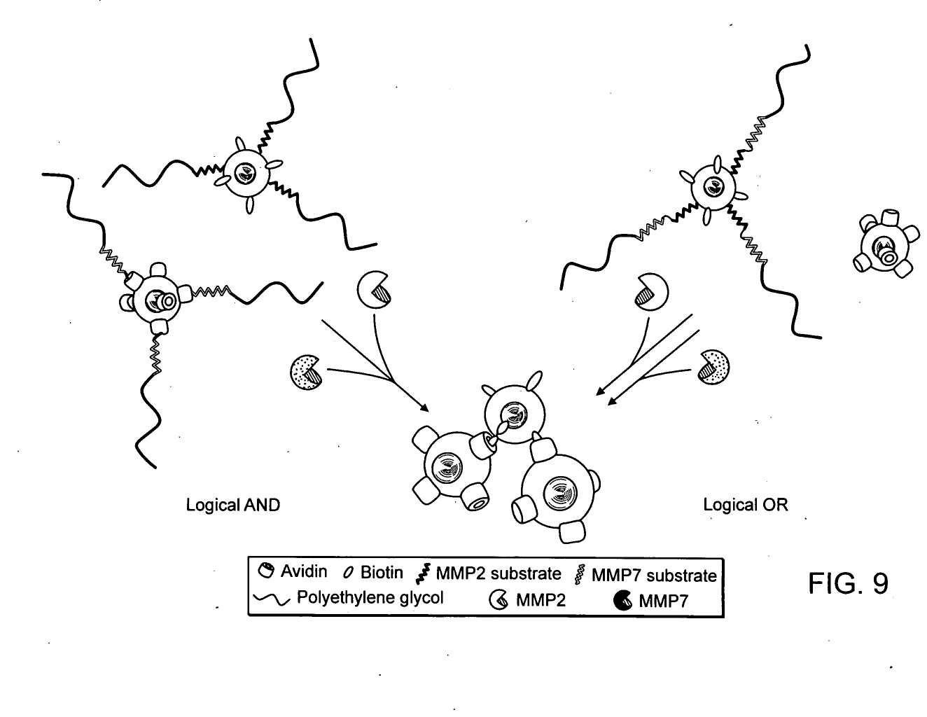

Accordingly, the present invention encompasses the recognition that by conjugating blocking agents to each TSAC via unique protease substrates, assembly can be restricted to occur only in the presence of both proteases (Logical “AND”; FIG. 9). Furthermore, by conjugating blocking agents to only the ligand TSAC with a tandem peptide substrate (containing both enzyme cleavage motifs in series), we sought to actuate assembly in the presence of either or both of the enzyme inputs (Logical “OR”; FIG. 9).

### “AND” TSACs

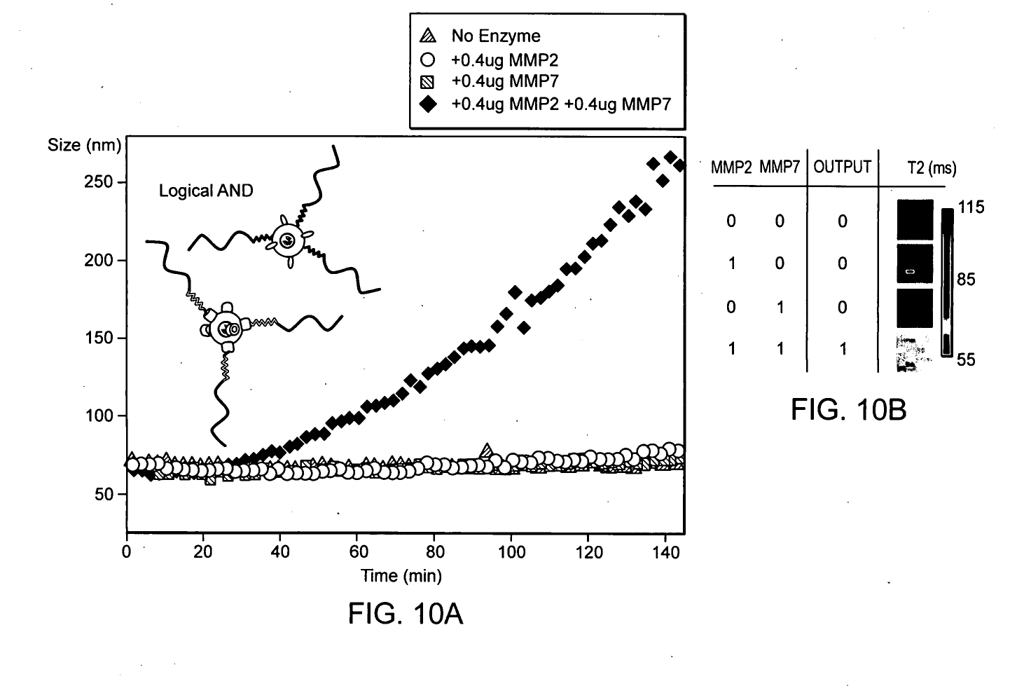

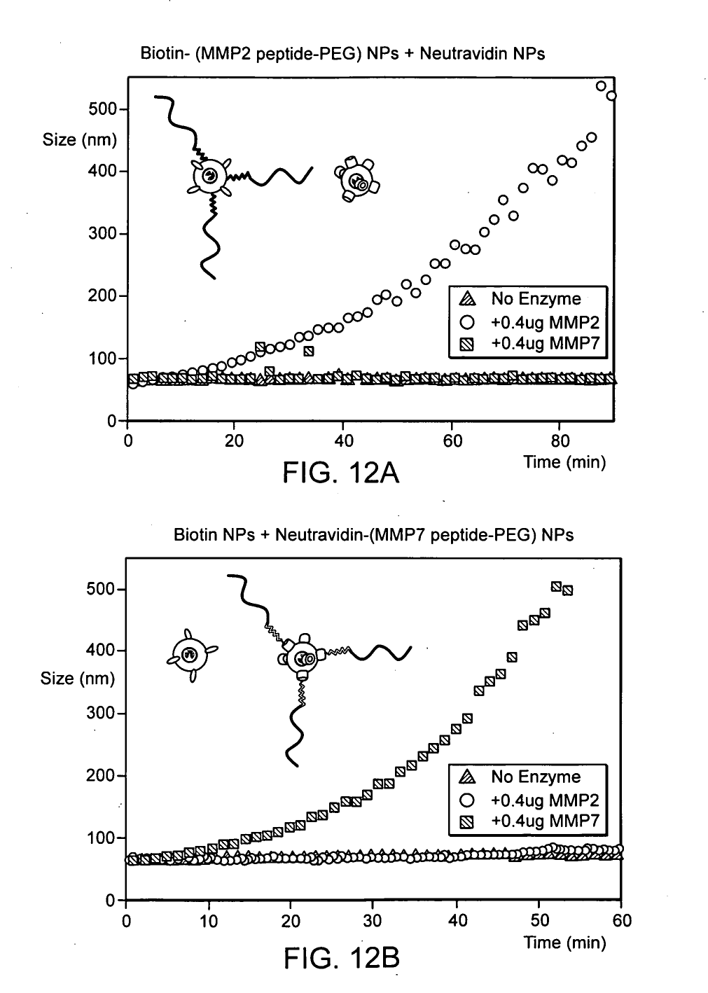

To begin “AND” TSAC synthesis, ligand TSACs were shielded with an MMP-2 (Gly-Pro-Leu-Gly-Val-Arg-Gly) (Bremer et al., 2001, Nat. Med., 7:743) substrate-PEG, and receptor particles were shielded with an MMP-7 (Val-Pro-Leu-Ser-Leu-Thr-Met) (Turk et al., 2001, Nat. Biotechnol., 19:661) substrate-PEG. Peptide-PEG conjugates were synthesized by reacting the peptide N-terminus (or lysine residue for “OR” tandem peptide) with an amine-reactive, 20 kDa methoxy-PEG-succimidyl α-methylbutanoate polymer. Cysteine residues were incorporated at the C-terminus of peptides to allow oriented attachment of substrate polymers onto nanoparticles. Specificity for these sequences was assessed by monitoring each enzyme's ability to actuate assembly of peptide-shielded particles in the presence of their unmodified cognate particles. In dynamic light scattering, specific enzyme-substrate pairs rapidly catalyzed the formation of nano- and micro-assemblies, while non-specific pairs negligibly affected population size (FIG. 12). By combining MMP-2-PEG ligand particles with MMP-7-PEG receptor particles, a logical “AND” system was created. Here, in presence of either protease alone, assembly of TSACs was prohibited by PEG polymers remaining on the cognate particle. In the presence of both proteases, however, TSAC assembly began and the population hydrodynamic radius increased 5-fold over 3 hours in dynamic light scattering (FIG. 10A). Further, assembled TSAC s were able to express “AND” logic in T2 relaxation changes, mapped using a 4.7 T Bruker MRI and Carr-Purcell-Meiboom-Gill pulse sequence. In the presence of both enzymes, T2 relaxation is enhanced by approximately 30% as compared to samples with no enzyme or either enzyme alone (FIG. 10B). This enhancement is comparable to published magnetic relaxation sensors (Perez et al., 2002, Nat. Biotechnol., 20:816; Perez et al., 2003, J. Am. Chem. Soc., 125:10192; and Harris et al., 2006, Angew Chem. Int. Ed. Engl., 45:3161), and occurs at MMP-2 concentrations that mimic tumor activity levels in vivo (2 μg MMP-2/ml=110 U/ml vs 435 U/g in vivo; Bremer et al., 2001, Nat. Med., 7:743).

### “OR” TSACs

A second system was constructed to actuate assembly in the presence of either of two proteolytic inputs (Logical “OR”). Again, ligand and receptor particles were synthesized, however, only the particles containing the ligand were masked with peptide-conjugated polymers. Here, a tandem MMP-2-MMP-7 peptide substrate was synthesized, containing both cleavage motifs in series (separated by an aminohexanioc acid spacer) to allow either enzyme to actuate assembly. Hydrodynamic radii increased more than 5-fold in the presence of either enzyme or both enzymes, indicating proper “OR” function (FIG. 11A). Accordingly, in the presence of either or both enzymes, “OR” TSAC T2 relaxation decreases approximately 40% as compared to samples with no enzyme (FIG. 11B).

### Discussion

In conclusion, the present invention demonstrates the synthesis of TSACs that use Boolean logic to simultaneously monitor multiple biological processes associated with tumorigenesis. The present invention encompasses the recognition that, in the future, logical TSAC switches may enable more informative imaging of neoplastic transformation in optically opaque samples both in vitro and in vivo. The modular design of these logical TSAC sensors can be applied to other enzymatic triggers, complimentary ligand/receptor pairs, or nanoparticle cores (semiconductor, plasmonic). Looking further, logical TSAC switches may enable specific localization of the processes underlying malignant transformation in vivo, as proteolytically-assembled beacons in sites of neoplastic inception. Such interstitial assembly may amplify the retention of particles (by mechanical entrapment in the tumor interstitium) and allow MRI visualization of diagnostic logic functions.

### EQUIVALENTS AND SCOPE

Those skilled in the art will recognize, or be able to ascertain using no more than routine experimentation, many equivalents to the specific embodiments of the invention, described herein. The scope of the present invention is not intended to be limited to the above Description, but rather is as set forth in the appended claims.

Those skilled in the art will recognize, or be able to ascertain using no more than routine experimentation, many equivalents to the specific embodiments of the invention described herein. The scope of the present invention is not intended to be limited to the above Description, but rather is as set forth in the appended claims.

In the claims articles such as “a,” “an,” and “the” may mean one or more than one unless indicated to the contrary or otherwise evident from the context. Thus, for example, reference to “a nanoparticle” includes a plurality of such nanoparticle, and reference to “the cell” includes reference to one or more cells known to those skilled in the art, and so forth. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process. Furthermore, it is to be understood that the invention encompasses all variations, combinations, and permutations in which one or more limitations, elements, clauses, descriptive terms, etc., from one or more of the listed claims is introduced into another claim. For example, any claim that is dependent on another claim can be modified to include one or more limitations found in any other claim that is dependent on the same base claim. Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise.

Where elements are presented as lists, e.g., in Markush group format, it is to be understood that each subgroup of the elements is also disclosed, and any element(s) can be removed from the group. It should it be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not been specifically set forth in haec verba herein. It is noted that the term “comprising” is intended to be open and permits the inclusion of additional elements or steps.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention (e.g., any monomeric unit, any complementary binding moiety, any blocking agent, any cleavable linker, any method of administration, any method of use, etc.) can be excluded from any one or more claims, for any reason, whether or not related to the existence of prior art.

The publications discussed above and throughout the text are provided solely for their disclosure prior to the filing date of the present application. Nothing herein is to be construed as an admission that the inventors are not entitled to antedate such disclosure by virtue of prior disclosure.

1-36. (canceled)

37. A self-assembly nanosystem, comprising:
a plurality of conjugates, wherein each conjugate comprises:
a biologically compatible monomeric unit,
at least one complementary binding moiety conjugated to the biologically compatible monomeric unit; and
at least one removably associated blocking agent, wherein the blocking agent shields the complementary binding moiety until the blocking agent is removed, and
wherein the monomeric unit, complementary binding moiety, and removably associated blocking agent are selected and arranged such that the conjugate adopts at least two relative configurations, a first relative configuration in which individual conjugates have not undergone self-assembly, and a second relative configuration in which individual conjugates have self-assembled to form an aggregate, wherein conversion from the first to the second relative configuration occurs in response to a trigger.

38. The self-assembly nanosystem of claim 37, wherein the biologically compatible monomeric unit is selected from the group consisting of a dendrimer, a nanoemulsion, a liposome, a polymer, a micelle, a protein, and a peptide.

39. The self-assembly nanosystem of claim 37, wherein the biologically compatible monomeric unit comprises a nanoparticle.

40. The self-assembly nanosystem of claim 37, wherein the biologically compatible monomeric unit comprises a microparticle.

41. The self-assembly nanosystem of claim 37, wherein the complementary binding moiety is selected from the group consisting of a ligand, an anti-ligand, a receptor, an antibody, an antigen, a phage-display derived peptide, a nucleic acid, an aptamer, a charge complex, and a reactive chemical moiety, and combinations thereof.

42. The self-assembly nanosystem of claim 37, wherein the complementary binding moiety is streptavidin.

43. The self-assembly nanosystem of claim 37, wherein the complementary binding moiety is biotin.

44. The self-assembly nanosystem of claim 37, wherein the removably associated blocking agent is selected from the group consisting of polaxamines; poloxamers; polyethylene glycol (PEG); peptides or other synthetic polymers of sufficient length and density to both mask self-assembly and provide protection against non-specific adsorption, opsonization, and RES uptake.

45. The self-assembly nanosystem of claim 37, wherein the blocking agent is conjugated to the monomeric unit or complementary binding moiety via a cleavable linker.

46. The self-assembly nanosystem of claim 37, wherein the monomeric unit further comprises a cargo entity.

47. The self-assembly nanosystem of claim 37, wherein the cargo entity is a diagnostic agent.

48. The self-assembly nanosystem of claim 47, wherein the diagnostic is selected from the group consisting of T2 contrast agent from the association of iron oxide nanoparticles; x-ray, optical, or ultrasound contrast from the periodic structure of an assembled aggregate; multi-modal imaging from the association of multiple imaging or contrast agents in a single aggregate; and combinations thereof.

49. The self-assembly nanosystem of claim 37, wherein the cargo entity is a therapeutic agent.

50. The self-assembly nanosystem of claim 49, wherein therapeutic is selected from the group consisting of activation of a drug from association of prodrug and activator carrying nanoparticles; activation of photo-dynamic therapy (PDT) from association of PDT and bioluminescent carrying nanoparticles; creation of single magnetic moment aggregates from the assembly of super-paramagnetic moment nanoparticles for subsequent targeting of super-paramagnetic nanoparticles to the diseased site; and combinations thereof.

51. The self-assembly nanosystem of claim 37, wherein all of the conjugates of the plurality of conjugates are identical to one another.

52. The self-assembly nanosystem of claim 37, wherein the plurality of conjugates comprises one or more populations of non-identical conjugates.

53. The self-assembly nanosystem of claim 52, wherein one population of non-identical conjugates comprises one complementary binding moiety, and another population of non-identical conjugates comprises a different complementary binding moiety.

54. The self-assembly nanosystem of claim 52, wherein one population of non-identical conjugates comprises one monomeric unit, and another population of non-identical conjugates comprises a different monomeric unit.

55. The self-assembly nanosystem of claim 52, wherein one population of non-identical conjugates comprises one blocking agent, and another population of non-identical conjugates comprises a different blocking agent.

56. The self-assembly nanosystem of claim 45, wherein all of the conjugates of the plurality of conjugates are identical to one another.

57. The self-assembly nanosystem of claim 45, wherein the plurality of conjugates comprises one or more populations of non-identical conjugates.

58. The self-assembly nanosystem of claim 57, wherein one population of non-identical conjugates comprises one cleavable linker, and another population of non-identical conjugates comprises a different cleavable linker.

59. The self-assembly nanosystem of claim 57, wherein one population of non-identical conjugates comprises one cargo entity, and another population of non-identical conjugates comprises a different cargo entity.

60-72. (canceled)

73. A method of treating a disease, condition, or disorder comprising administering the self-assembly nanosystem of claim 37 to a subject.

74. The method of claim 73, wherein the disease, condition, or disorder is a cell proliferative disorder.

75. The method of claim 73, wherein the disease, condition, or disorder is cancer.

76. The method of claim 73, wherein the disease, condition, or disorder has an inflammatory component.

77-81. (canceled)
